SEQUENCE LISTING (13 sequences)

SEQ ID NO: 1 - RNA, 100 nt, Homo sapiens
  source 1..100: mol_type = mRNA
tgtccaggcgtggaccgctctgccacgcactagctcggttattggttatgccaaaggcactctccatctcccacatctggttattgacaagtgtaacttt

SEQ ID NO: 2 - RNA, 100 nt, Homo sapiens
  source 1..100: mol_type = mRNA
tctaatactcctcctctaacatgtcagcgttattgtaatgcaagtgtgaccaattcagtgaaaggaacgaatgcgattctctggacctgtttgggactga

SEQ ID NO: 3 - RNA, 100 nt, Homo sapiens
  source 1..100: mol_type = mRNA
tagatagtatggtcaacaataatataaggaagaaagatcatgaacccctacgagctgtgtgctgtagcctcattaattggttagaagcaaacgctgaata

SEQ ID NO: 4 - RNA, 100 nt, Homo sapiens
  source 1..100: mol_type = mRNA
ttctcaggacaccatccgttcaattggtacaaagctggtatatccagagtcttcaggagcttcttgattttaaggacaaaagtgctgaggatgctaaagc

SEQ ID NO: 5 - RNA, 100 nt, Homo sapiens
  source 1..100: mol_type = mRNA
tgtccctgggcctcggcctcctgctgctggtcatcatctgtgtggttggattccaaaattccaaatttcagagggacctggtgaccctgagaacagattt

SEQ ID NO: 6 - RNA, 100 nt, Homo sapiens
  source 1..100: mol_type = mRNA
tttgcattctggggtgacactgctgtagagaggttgaacgtgttcattaccatggccaaggtctttctgatcctccacttcattattggcttcagcgtgc

SEQ ID NO: 7 - RNA, 100 nt, Homo sapiens
  source 1..100: mol_type = mRNA
gagaaagtggattgaacaaggacgcatttccccagtacatccacaacatgctgtccacatctcgttctcggtttatcagaaataccaacgagagcggtga

SEQ ID NO: 8 - RNA, 100 nt, Homo sapiens
  source 1..100: mol_type = mRNA
tttcagccctgccgagtcctcttgtgacctcaggaaagagcgtgaccctgctgtgtcagtcacggagcccaatggacacttttcttctgatcaaggagcg

SEQ ID NO: 9 - RNA, 100 nt, Homo sapiens
  source 1..100: mol_type = mRNA
catgaaatgtctggtctgtccaccccatcaacaagtcttgaaacaagcaacagatggatagtctgtccaaatggacataagacagacagcagtttccctg

SEQ ID NO: 10 - RNA, 100 nt, Homo sapiens
  source 1..100: mol_type = mRNA
accatttgtctttagtcttggcaaaggccaagtcatcaaggcatgggacattggggtggctaccatgaagaaaggagagatatgccatttactgtgcaaa

SEQ ID NO: 11 - RNA, 100 nt, Homo sapiens
  source 1..100: mol_type = mRNA
aaaacataccaattgttggccagaagggaagtctgggatcaaagtgctttggatgttgggttaacaatgcagctcctggactgcaacttctcagatgaaa

SEQ ID NO: 12 - RNA, 100 nt, Homo sapiens
  source 1..100: mol_type = mRNA
gctgtaacttcctagagttgcaccctagcaacctagccagaaaagcaagtggcaagaggattatggctaacaagaataaatacatgggaagagtgctccc

SEQ ID NO: 13 - RNA, 100 nt, Homo sapiens
  source 1..100: mol_type = mRNA
actgattatacctctagtatagatgtatggtctgctggctgtgtgttggctgagctgttactaggacaaccaatatttccaggggatagtggtgtggatc

(19) United States
(12) Patent Application Publication
(10) Pub. No.: US 2024/0091385 A1
(43) Pub. Date: Mar. 21, 2024

(54) METHODS OF ASSESSING SELECTIVE GLUCOCORTICOID RECEPTOR MODULATION AND OF IDENTIFYING AND TREATING PATIENTS LIKELY TO BENEFIT FROM GLUCOCORTICOID RECEPTOR MODULATION

(71) Applicant: Corcept Therapeutics Incorporated, Menlo Park, CA (US)

(72) Inventors: Andrew Greenstein, San Francisco, CA (US); Hazel Hunt, Storrington (GB)

(73) Assignee: Corcept Therapeutics Incorporated, Menlo Park, CA (US)

(21) Appl. No.: 18/460,226

(22) Filed: Sep. 1, 2023

Related U.S. Application Data
(60) Provisional application No. 63/403,560, filed on Sep. 2, 2022.

(51) Int. Cl. A61K 49/00; A61K 31/337; A61K 31/4166; A61K 31/444; A61P 35/00; C12Q 1/6874; C12Q 1/6886

(52) U.S. Cl. CPC A61K 49/0004; A61K 31/337; A61K 31/4166; A61K 31/444; A61P 35/00; C12Q 1/6874; C12Q 1/6886; C12Q 2600/106; C12Q 2600/158

(57) ABSTRACT

Combined administration of a selective glucocorticoid receptor modulator (SGRM; e.g., relacorilant or exicorilant) and a cancer therapeutic (e.g., a taxane or antiandrogen) is useful for identifying elevated cortisol activity in patients with cancer or with Cushing's syndrome, and for treating patients with cancer (e.g., ovarian, pancreatic, or prostate cancer). An at least 40% change in RNA levels encoding CDKN1C, TNFRSF17, BRIP1, PDK1, CLEC10A, FPR3, CCR2, LTLRB4, or CD86 indicates that the patient with cancer is likely to benefit from the combined treatment (e.g., likely to have longer survival than similar patients not receiving combined treatment). An active SGRM dose is identified where RNA levels encoding CDKN1C, TNFRSF17, BRIP1, or PDK1 decrease, or where RNA levels encoding CLEC10A, FPR3, CCR2, LTLRB4, and CD86 increase, by at least 40%. Changes in RNA levels encoding CLEC10A, FPR3, CCR2, LTLRB4, and CD86 identify patients with cancer or with Cushing's having elevated cortisol activity.

### CROSS-REFERENCE TO RELATED APPLICATIONS

This application claims priority to, and the benefit of, U.S. Provisional Application No. 63/403,560, filed Sep. 2, 2022, which application is hereby incorporated by reference herein in its entirety.

### REFERENCE TO A “SEQUENCE LISTING,” A TABLE, OR A COMPUTER PROGRAM LISTING APPENDIX SUBMITTED ON A COMPACT DISK

The instant application contains a Sequence Listing which has been submitted electronically in XML format and is hereby incorporated by reference in its entirety. Said XML copy, created on Aug. 24, 2023, is named 081906-1326635-171840US_SL.xml and is 13,367 bytes in size.

### BACKGROUND

Cortisol is the most abundant endogenous agonist of the glucocorticoid receptor (GR) in humans. Cortisol may be measured, for example, in blood, urine, saliva, or tear samples; however, the cortisol levels measured in these different samples differ and do not correlate with each other. Urinary free cortisol (a measure of cortisol in urine excreted over 24 hours) is used to diagnose Cushing's syndrome; in addition, plasma cortisol (a measure of the cortisol levels at the time the blood sample is taken) is used for dexamethasone suppression testing (which tests patient response to rapid increases in glucocorticoid levels). In addition, cortisol levels can be measured in a whole blood or serum sample according to methods known in the art.

Cortisol levels vary during the day, with higher levels in the morning, and low levels at night. Normal morning serum levels are sufficient to activate GR systemically [Wang & Harris, Adv Exp Med Biol. (New York, NY: Springer New York). 2015; 2895-98.]. Study of GR physiology would be aided by selective glucocorticoid receptor modulators (SGRMs). GR-specific agonists, such as dexamethasone, are capable of probing super-physiological GR activation [Shen et al., Archives of Surgery, 141, 771 (2006)]. While endogenous GR agonists (ie, cortisol and corticosterone) are abundant in mammals, there is no naturally occurring GR antagonist to complicate the interpretation of systemic SGRM effects on gene transcription. Steroidal GR antagonists, however, which are limited to mifepristone and its analogs, lack specificity for GR [Rew et al., Journal of Medicinal Chemistry, 61, 7767-7784 (2018)]. Thus, there is need for GR-specific antagonists for use in elucidating the biological role of endogenous cortisol by specifically antagonizing cortisol activity on the GR.

Nuclear hormone receptors represent a rare example of druggable transcriptional regulators [Frigo et al., Essays in Biochemistry, 65, 847-856 (2021)]. Selective androgen and estrogen receptor modulators (SARMs and SERMs) are important medicines in oncology and in the treatment of endocrine disorders. SGRMs are an emerging class of promising drug candidates in oncology, endocrine, and metabolic diseases [Munster et al., Clinical Cancer Research, 28, 3214-3224 (2022); Colombo et al., Journal of Clinical Oncology, 40, LBA5503-LBA5503 (2022)]. Thousands of target genes for the GR have been reported in various contexts [Greenstein et al., Endocrine-Related Cancer, 28, 583-592 (2021)], and published studies commonly report short-term effects of GR agonists and homogenous populations of cells in vitro [McDowell et al., Genome Research, 28, 1272-1284 (2018); Arora et al., Cell, 155, 1309-1322 (2013); Stringer-Reasor et al., Gynecologic Oncology, 138, 656-662 (2015)]. Many of the reported GR responsive genes are also AR and ER target genes, underscoring the complexity of nuclear hormone signaling [Arora et al., Cell, 155, 1309-1322 (2013); and Isikbay et al., Hormones and Cancer, 5, 72-89 (2014)]. Previous studies have not addressed the broader role of cortisol signaling in the human body via identification of GR-specific target genes systemically.

Accordingly, there is a need for identification of genes regulated by GR, and for novel therapeutic options for modulating GR for therapy for disorders of GR regulation and other disorders, including cancer and Cushing's syndrome.

### SUMMARY

Disclosed herein are novel methods for identifying patients likely to benefit from therapy including administration of a selective glucocorticoid receptor modulator (SGRM), for identifying an active dose of such a SGRM in a patient with cancer, and for identifying patients with cancer with relatively elevated cortisol activity as compared to other similar patients with cancer. In embodiments, the methods include combined cancer therapies comprising administering a SGRM and a cancer therapeutic agent, and methods for identifying patients likely to benefit from such combined therapies. In embodiments, the SGRM inhibits GR activation. In embodiments, the SGRM is a non-steroidal SGRM, and may be a heteroayl-ketone fused azadecalin or an octahydro fused azadecalin. In embodiments, the SGRM is selected from relacorilant and exicorilant. The cancer therapeutic agent may be, e.g., a taxane, an antiandrogen, or other cancer therapeutic agent. In embodiments, the taxane is paclitaxel or nab-paclitaxel. In embodiments, the antiandrogen is enzalutamide. In embodiments, administration of a SGRM and a cancer therapeutic (e.g., a taxane or an antiandrogen) is effective to increase the survival of the patient (i.e., the patient survives for a longer time after combined SGRM and cancer therapeutic treatment, than would be expected for such a patient receiving the cancer therapeutic alone). In embodiments, the likely benefit of such combined therapy includes increased survival of the patient. In embodiments, the cancer patient suffers from a cancer selected from ovarian, pancreatic, and prostate cancer.

Also disclosed herein are novel methods for identifying an active dose of a SGRM in a patient with Cushing's syndrome. In embodiments, the SGRM inhibits the activation of the glucocorticoid receptor (GR). In embodiments, the SGRM is relacorilant or is exicorilant.

Methods disclosed herein include methods of assessing the pharmacodynamic effects of SGRMs, and of identifying a patient with cancer likely to benefit from SGRM-containing therapies, comprising administering a SGRM and a cancer therapeutic agent (e.g., a taxane such as, e.g., paclitaxel or nab-paclitaxel; an antiandrogen, such as, e.g., enzalutamide, or other cancer therapeutic agent), and measuring, as compared to baseline, changes in systemic RNA levels (e.g., as measured in blood samples, e.g., whole blood samples) of RNAs encoding one or more of CDKN1C, TNFRSF17, BRIP1, PDK1, CLEC10A, FPR3, CCR2, LTLRB4, and CD86 in the patient. In embodiments, the RNA levels are whole blood RNA levels. In embodiments, a change of RNA level of at least 40% (as compared to a similar patient with cancer not receiving administration of a SGRM and a cancer therapeutic agent) in a patient with cancer receiving administration of a SGRM and a cancer therapeutic indicates that the patient will likely survive longer than a similar cancer patient not receiving administration of a SGRM and a cancer therapeutic. In embodiments, a decrease of at least 40% in the level of an RNA that encodes CDKN1C, TNFRSF17, BRIP1, or PDK1, or an increase of at least 40% in the level of an RNA that encodes CLEC10A, FPR3, CCR2, LTLRB4, or CD86 (as compared to a similar cancer patient not receiving administration of a SGRM and a cancer therapeutic agent) in a patient with cancer receiving administration of a SGRM and a cancer therapeutic indicates that the patient will likely survive longer than a similar cancer patient not receiving administration of a SGRM and a cancer therapeutic. In embodiments, the cancer patient suffers from a cancer selected from ovarian cancer, pancreatic cancer, and prostate cancer.

In embodiments, administration of a SGRM and a cancer therapeutic is effective to increase the survival of the patient beyond the survival expected for that patient in the absence of SGRM administration, where the survival expected for that patient in the absence of SGRM administration is the survival expected for such a patient receiving a cancer therapeutic alone, without concomitant SGRM administration. In embodiments, the cancer therapeutic may be a taxane (e.g., paclitaxel or nab-paclitaxel), may be the antiandrogen enzalutamide, or may be a different cancer therapeutic.

Applicant further discloses herein methods of using changes from baseline in RNA levels encoding one or more of CDKN1C, TNFRSF17, BRIP1, PDK1, CLEC10A, FPR3, CCR2, LTLRB4, and CD86 for guiding identification of an active dose of SGRM to be administered to a patient in need of SGRM administration. In embodiments, an SGRM dose that elicits a decrease in the level of an RNA that encodes CDKN1C, TNFRSF17, BRIP1, or PDK1, or that elicits an increase in the level of an RNA that encodes CLEC10A, FPR3, CCR2, LILRB4, or CD86 (as compared to baseline) is identified as an active dose of the SGRM. In embodiments, the decrease, or increase, that identifies an SGRM dose as an active dose is a decrease, or increase, of at least 40% as compared to baseline. In embodiments, the identified active SGRM dose is administered to a patient along with administration of a cancer therapeutic.

Applicant further discloses herein methods of using baseline RNA levels encoding one or more of CLEC10A, FPR3, CCR2, LTLRB4, CD86, FKBP5, GSK3B, PIK3CG, and MCL1 to identify patients with cancer with elevated cortisol activity (as compared to similar patients with cancer). In embodiments, the cancer patient suffers from a cancer selected ovarian cancer, pancreatic cancer, and prostate cancer.

In embodiments, in a patient with Cushing's syndrome receiving administration of a dose of a SGRM, a change of RNA level of at least 40% (as compared to the baseline level of that RNA in that patient with Cushing's syndrome) indicates that the dose of SGRM is an active dose.

In embodiments, the SGRM is relacorilant (also known as CORT125134) or is exicorilant (also known as CORT125281). Relacorilant is a heteroaryl-ketone fused azadecalin compound having the chemical name (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone; it has the following structure:

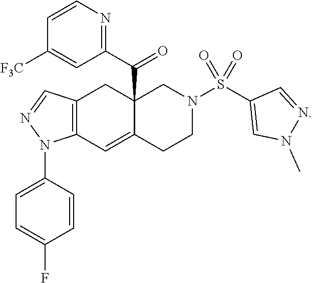 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Exicorilant is an octahydro fused azadecalin compound having the chemical name ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone; it has the following structure:

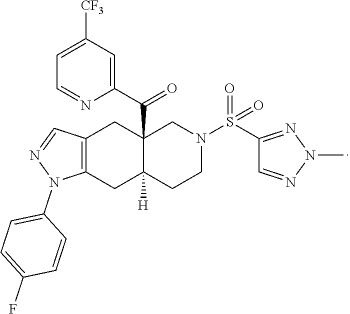 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

Accordingly, Applicant discloses herein improved methods for identifying and treating patients with cancer who would benefit from combined SGRM and cancer therapeutic treatment; for identifying active doses of SGRM; for identifying and treating patients with cancer with excess cortisol activity; for identifying active SGRM doses for such treatments; for increasing overall survival in patients with cancer; methods for identifying active SGRM doses for treating patients with Cushing's syndrome; methods for treating patients with Cushing's syndrome with those SGRM doses; and other beneficial diagnostic and treatment methods.

### BRIEF DESCRIPTION OF THE DRAWINGS

FIG. 1A. Design of the randomized open-label phase 2 ovarian cancer study. Patients received nab-paclitaxel (NP) alone or with relacorilant (dosed either continuously or intermittently). Patients that progressed on nab-paclitaxel alone were allowed to cross over to NP plus continuous relacorilant.

FIG. 1B. Design of the randomized open-label phase 2 ovarian cancer study. RNA sampling (red triangles) occurred pre-dose on cycle 1 day 1 and day 15. This captured the pharmacodynamic effects of 14 consecutive days of relacorilant dosing for patients on the continuous arm (not shown). For patients on the intermittent arm, however, this effectively represents sampling after a washout (days 10-13) followed by a single relacorilant dose (day 14) (black diamonds represent relacorilant dosing days). Granulocyte colony stimulating factor (G-CSF) (grey bars) was mandated for all patients receiving relacorilant at days 2, 9, and 16 of each cycle.

FIG. 2A illustrates the scheme used for identifying the 444 target genes that distinguish patients treated with or without relacorilant.

FIG. 2B. Genes identified that distinguish patients treated with or without relacorilant. Machine learning distinguishes pharmacodynamic effects of relacorilant+NP (combined continuous and intermittent arms) versus (“vs”) NP alone based on fold change in a 444 gene panel. Genes were measured at C1D1 and C1D15, and fold change calculated for each gene. Cross-validated random forest methods identified a set of genes whose fold changes were reliably different between relacorilant+nab-paclitaxel vs nab-paclitaxel alone (FIG. 2B, left). As a control, the baseline values of these genes did not predict treatment arm (FIG. 2B, right).

FIG. 3. Fold change in 8 genes that were associated with relacorilant activity The top genes that were important in distinguishing study arms identified in FIG. 2 were prioritized based on the adjusted significance in a t-test comparing fold change in relacorilant+nab-paclitaxel vs nab-paclitaxel alone. The fold change in each gene for each subject is represented by a data point after treatment with (A) continuous relacorilant+NP, (B) intermittent relacorilant+NP, or (C) NP alone.

FIG. 4. Fold change in key genes before and after crossover from NP alone to relacorilant+NP For patients that crossed over from nab-paclitaxel alone to continuous relacorilant+NP, the changes in 4 key genes are shown before (left side of each graph) and after (right side of each graph) the crossover.

FIG. 5A. Cross-study pharmacodynamic analysis of 8 key genes. The same analysis of log 2 fold change in the 8 genes identified in the randomized phase 2 ovarian trial was applied to other SGRM clinical studies. The study illustrated in 5A was a study of continuous relacorilant+NP in pancreatic ductal adenocarcinoma.

FIG. 5B. Cross-study pharmacodynamic analysis of 8 key genes. The study illustrated in 5B was a study of continuous or intermittent relacorilant+NP in various solid tumors.

FIG. 5C. Cross-study pharmacodynamic analysis of 8 key genes. The study illustrated in 5C was a study of continuous relacorilant+enzalutamide in prostate cancer.

FIG. 5D. Cross-study pharmacodynamic analysis of 8 key genes. The study illustrated in 5D was a study of continuous exicorilant+enzalutamide in prostate cancer.

FIG. 5E. FIG. 5E illustrates CDKN1C expression levels at baseline and after two weeks of exicorilant+enzalutamide administration in prostate cancer patients.

FIG. 6A. CLEC10A is suppressed by GR agonists. 25 mg prednisone suppressed CLEC10A in healthy volunteers.

FIG. 6B. CLEC10A is suppressed by GR agonists. Subjects with low baseline CLEC10A had high 24 hr UFC, and high baseline CLEC10A was associated with lower 24 hour urinary free cortisol (UFC).

FIG. 7A. Induction of CLEC10A by relacorilant is associated with improved overall survival in ovarian cancer. The fold change from C1D1 to C1D15 in CLEC10A for all subjects in the ovarian phase 2 study receiving continuous relacorilant+NP was determined. Patients with fold change values in the upper two tertiles (CLEC10A induced, black) had longer overall survival as compared to patients with fold changes values in the lower tertile (CLEC10A not induced, gold). Overall survival of all subjects (regardless of CLEC10A induction) treated with NP alone is shown as a comparator (black).

FIG. 7B. Induction of CLEC10A by relacorilant is associated with improved overall survival in ovarian cancer. In a large public database of ovarian cancer tumor gene expression, patients with baseline CLEC10A in the upper two tertiles (red) had longer overall survival as compared to patients with baseline CLEC10A in the lower tertile (black).

### DETAILED DESCRIPTION

### Introduction

Methods and discoveries disclosed herein include methods of identifying a patient with cancer likely to benefit from administration of a SGRM and a cancer therapeutic; methods of treating the identified patient; methods of identifying a patient with cancer as having elevated cortisol activity, and for treating that identified cancer patient; methods for identifying an active dose of a SGRM in a patient with cancer; methods of identifying an active dose of a SGRM in a patient with Cushing's syndrome; and other beneficial diagnostic and treatment methods.

The methods disclosed herein can be used to identify, and can be used to treat, patients with cancer who would benefit from combined treatment with a selective glucocorticoid receptor modulator (SGRM) and a cancer therapeutic (cancer therapy treatment). Such cancer therapeutic treatments (cancer therapies) may include, taxane treatment (e.g., paclitaxel or nab-paclitaxel treatment); antiandrogen treatment (e.g., enzalutamide treatment), and other cancer therapies. Such patients with cancer include patients with cancer having excess cortisol activity; and include patients with cancer suffering from, e.g., ovarian, pancreatic, or prostate cancer. Methods disclosed herein include methods for identifying active SGRM doses for such treatments, and for increasing survival time, after treatment, in cancer patients (as compared to the expected survival time for patients not receiving such treatment; often termed “longer overall survival”). Active doses identified by the methods disclosed herein are believed to be effective doses useful for treating patients with cancer. Benefits to patients with cancer receiving such combined SGRM and cancer therapeutic treatments include, for example, increased length of survival after treatment, as compared to similar patients with cancer not receiving such treatment. SGRMs useful in these methods include heteroaryl-ketone fused azadecalin compounds such as relacorilant and include octahydro fused azadecalin compounds such as exicorilant. In embodiments, the SGRM is a selective glucocorticoid receptor antagonist (SGRA).

The methods disclosed herein further include identifying active SGRM doses for treating patients with Cushing's syndrome, and for treating patients with Cushing's syndrome. Active doses identified by the methods disclosed herein are believed to be effective doses useful for treating patients with Cushing's syndrome. The patients with Cushing's syndrome may suffer from Cushing's Disease. SGRMs useful in these methods include heteroaryl-ketone fused azadecalin compounds such as relacorilant and include octahydro fused azadecalin compounds such as exicorilant. In embodiments of these methods directed to patients with Cushing's syndrome, the SGRM is a SGRA.

SGRMs represent a new tool for interrogating basic glucocorticoid receptor (GR) function and cortisol activity at the GR [Greenstein and Hunt, Oncotarget, 12, 1243-1255 (2021)]. Cortisol is the most abundant endogenous GR agonist in humans, and normal morning serum levels are sufficient to activate GR systemically [Wang & Harris, Adv Exp Med Biol. (New York, NY: Springer New York). 2015; 2895-98]. Steroidal GR antagonists, such as mifepristone and its analogs, lack specificity for GR alone [Rew et al., Journal of Medicinal Chemistry, 61, 7767-7784 (2018)]. The compounds used in the Examples disclosed herein, relacorilant and exicorilant, are non-steroidal SGRMs that selectively inhibit GR activity with no affinity for other hormone receptors. [Hunt et al., Journal of Medicinal Chemistry, 60, 3405-3421 (2017)]. GR-specific agonists, such as dexamethasone, are capable of probing super-physiological GR activation [Shen et al., Archives of Surgery, 141, 771 (2006)]. While endogenous GR agonists (i.e., cortisol and corticosterone) are abundant in mammals, there is no naturally occurring GR antagonist to complicate the interpretation of systemic SGRM effects on gene transcription. Established SGRM safety [Munster et al., Clinical Cancer Research, 28, 3214-3224 (2022); Pivonello et al., Frontiers in Endocrinology, 12, 1-12 (2021)] allows dosing on the scale of weeks to years, while most existing reports focus on the short-term effects of GR (on the scale of hours to days) [Olnes et al., Scientific Reports, 6, 23002 (2016); and Stringer-Reasor et al., Gynecologic Oncology, 138, 656-662 (2015); Al-Hity et al., Communications Biology, 4, 781 (2021)]. Thus, SGRMs are uniquely positioned to probe the biological role of endogenous cortisol by specifically antagonizing cortisol activity on the GR.

Applicant discloses herein specific transcriptional effects of SGRMs that have been identified in whole blood. Eight genes indicative of SGRM activity were identified in an ovarian cancer study of patients with ovarian cancer treated with nab-paclitaxel (NP) and relacorilant (relacorilant+NP). The specificity of these genes is underscored by their consistent performance across patients with distinct tumor types, concomitant medications, and even the co-administration of a selective androgen modulator (SARM; e.g., enzalutamide). The CLEC10A gene is a member of this gene panel, which is both acutely induced by super-physiological GR agonist and suppressed by GR antagonist in whole blood. Further, CLEC10A induction by SGRM predicts cancer patient overall survival after administration of relacorilant+NP therapy. Bioinformatic analysis indicates that CLEC10A and its correlates (FPR3, CCR2, LILRB4, and CD86) are markers of a set of dendritic cells. Together, these data elucidate new, clinically relevant, biological consequences of GR modulation in humans

SGRM administration was found to increase not only the amount of mRNA encoding CLEC10A in whole blood, but also to increase the amounts of mRNA encoding FPR3, CCR2, LTLRB4, and CD86 as well. Thus, discussion of increasing CLEC10A expression by SGRM administration also indicates not only the increase of CLEC10A mRNA levels, but also indicates increases in mRNA encoding FPR3, CCR2, LILRB4, and CD86.

SGRMs represent a clinically validated mechanism of enhancing chemosensitivity and chemotherapy efficacy and improving the sequelae of hypercortisolism. While GR reportedly controls expression of some 3000 genes, it is unknown which of these are specific targets of systemic GR activity in humans. Applicant established a robust assay to measure candidate GR target genes in human blood and assessed them before and after therapy with oral SGRMs that specifically antagonize the GR. Changes in these genes were assessed after treatment with SGRM plus nab-paclitaxel (NP) (compared to NP alone to eliminate changes due to disease state or NP therapy) in a randomized phase 2 ovarian cancer study (NCT03776812). Machine-learning identified a set of genes that accurately identified patients that had received SGRM (ROC AUC 0.945+/−0.038, where “ROC” means receiver operator characteristic curve, and “AUC” means are under the ROC). In patients that crossed over from NP alone to SGRM+NP these same genes were modified only after crossover within a given patient (paired t-test P=0.0003). These genes were found to be reliable indicators of SGRM activity across clinical studies in pancreatic, prostate, and other solid tumors (NCT04329949, NCT03437941, NCT03674814), independent of concomitant medications or other hematological confounders. CLEC10A appeared to be a particularly sensitive marker of GR activity as its expression was induced by SGRM in patients with solid tumors (paired t-test P<0.00010), suppressed by prednisone in healthy volunteers (paired t-test P<0.00010, NCT03335956), and suppressed in patients with high 24-hr urinary free cortisol (non-parametric t-test P=0.037). CLEC10A induction in blood by SGRM was associated with longer overall survival in patients with ovarian cancer treated with SGRM+NP (HR=0.39, Cox PH P=0.0135), and high baseline tumor CLEC10A expression was associated with longer OS in a large tumor ‘omics’ database (HR 0.8, log-rank P=0.0008, where “HR” indicates “Hazard Ratio”). CLEC10A and the genes correlated with its expression in these data sets are primarily expressed by a specific subset of dendritic cells. Analysis of CLEC10A and related SGRM-responsive genes confirms that systemic GR activity in patients with solid tumors can be modulated pharmacologically, indicates GR modulation can impact survival in patients with ovarian cancer, and provides new insight into the systemic functions of GR in humans.

Applicant discloses herein methods of assessing the pharmacodynamic effects of SGRMs using a set of genes in whole blood in patients with cancer, including in patients with cancer with ovarian, pancreas, or prostate tumors. Applicant discloses herein methods of identifying patients for whom treatment with a SGRM and a cancer therapeutic (e.g., a taxane or an antiandrogen) are likely to experience longer survival than patients not receiving such treatment. Applicant discloses herein methods of predicting which patients with cancer are likely to benefit from SGRM-containing therapies with administration of a SGRM and a cancer therapy (e.g., a taxane such as paclitaxel or nab-paclitaxel; an antiandrogen such as enzalutamide; or other cancer therapy) by measuring initial change in systemic RNA levels (e.g., as measured in whole blood samples) of RNAs encoding one or more of CDKN1C, TNFRSF17, BRIP1, PDK1, CLEC10A, FPR3, CCR2, LILRB4, and CD86 in the patient. In embodiments, the cancer patient suffers from a cancer selected ovarian cancer, pancreatic cancer, and prostate cancer. In embodiments, the SGRM is relacorilant. In embodiments, the SGRM is relacorilant and the taxane is nab-paclitaxel (termed “relacorilant+nab-paclitaxel”). In embodiments, the SGRM is exicorilant. In embodiments, the SGRM is exicorilant and the taxane is nab-paclitaxel (termed “exicorilant+nab-paclitaxel”).

Applicant discloses herein methods of using baseline RNA levels encoding one or more of CLEC10A, FKBP5, GSK3B, PIK3CG, and MCL1 to identify patients with elevated cortisol activity. In embodiments, the RNA levels are whole blood RNA levels. In embodiments, patients with elevated cortisol activity include patients with cancer and patients with Cushing's syndrome.

Applicant discloses herein methods of using change from baseline, following SGRM administration, in RNA levels encoding one or more of CDKN1C, TNFRSF17, BRIP1, PDK1, CLEC10A, FPR3, CCR2, LTLRB4, and CD86 as a pharmacodynamic biomarker for aiding in the determination of, or for determining, an active dose of SGRM to be administered to a patient in need of SGRM administration, where baseline RNA levels are measured prior to SGRM administration. In embodiments, the RNA levels are whole blood RNA levels. In embodiments, patients in need of SGRM administration include patients with cancer and patients with Cushing's syndrome (“patients with Cushing's syndrome”, and “Cushing's syndrome patients”, as the terms are used herein, include those suffering from Cushing's Disease, subclinical Cushing's syndrome, and difficult-to-diagnose Cushing's syndrome). In embodiments, administration of one dose level of SGRM leads to a response indicating that the dose level is an active dose. In embodiments, one or more dose levels administered to a patient do not lead to a response in the patient; such dose levels are not active dose levels. In such cases, further doses may be administered to the patient. For example, two or more further SGRM doses, each different than previously administered dose level(s), are administered in order to identify a SGRM dose that leads to a response in the patient.

Applicant discloses herein methods of using change from baseline, following SGRM administration, in RNA levels encoding one or more of CDKN1C, TNFRSF17, BRIP1, PDK1, CLEC10A, FPR3, CCR2, LTLRB4, and CD86 as a predictive biomarker to identify patients with cancer likely to benefit from treatments with SGRM plus cancer therapeutics as compared to treatment with the cancer therapeutic alone. In embodiments, the cancer therapy includes administration of a cancer therapeutic that is a taxane, or is an antiandrogen, or by administration of another cancer therapeutic or therapy. In embodiments, the RNA levels are whole blood RNA levels. Baseline RNA levels are measured prior to SGRM administration. In embodiments, the SGRM is relacorilant or exicorilant. In embodiments, the taxane is paclitaxel or nab-paclitaxel. In embodiments, the antiandrogen is enzalutamide. In embodiments, the cancer patient suffers from a cancer selected from ovarian, pancreatic, and prostate cancer.

Applicant discloses herein methods of increasing, in a patient with cancer, RNA levels encoding one or more of CLEC10A, FPR3, CCR2, LTLRB4, and CD86 comprising administering an effective dose of a SGRM to the patient. Applicant discloses herein methods of decreasing, in a patient with cancer, RNA levels encoding one or more of CDKN1C, TNFRS17, BRIP1, and PDK1 comprising administering an effective dose of a SGRM to the patient. In embodiments, the RNA levels are whole blood RNA levels. In embodiments, the SGRM is selected from relacorilant and exicorilant. In embodiments, the cancer patient suffers from a cancer selected from ovarian, pancreatic, and prostate cancer. In embodiments, the survival of the patient is increased beyond the survival expected for that patient in the absence of SGRM administration. In embodiments, the cancer patient is administered a SGRM along with a taxane (e.g., paclitaxel or NP), and the survival expected for that patient in the absence of SGRM administration is the survival expected for such a patient receiving a taxane (e.g., paclitaxel or NP) alone, without concomitant SGRM administration.

It is believed that increased levels of whole blood RNA encoding CLEC10A indicate increased numbers of a specific set of dendritic cells. It is further believed that increased numbers of a specific set of dendritic cells are indicative of an enhancement in the immune response useful for treating cancer in a patient in need of cancer treatment. In embodiments, the specific set of dendritic cells are dendritic cells expressing CLEC10A. Thus, Applicant discloses herein methods of detecting indications of increased numbers of a specific set of dendritic cells (e.g., dendritic cells expressing CLEC10A) in a patient with cancer. It is believed that such indications of increased numbers of a specific set of dendritic cells (e.g., dendritic cells expressing CLEC10A) are indicative of an enhancement in the immune response useful for treating cancer in a patient, and are indicative of a beneficial effect of treatment of the cancer. In embodiments, the cancer patient suffers from a cancer selected from ovarian, pancreatic, and prostate cancer.

Applicant further discloses herein methods, in a patient with cancer, using change from baseline in levels of RNA encoding CLEC10A as a biomarker to identify patients with increased numbers of a specific set of dendritic cells (e.g., dendritic cells expressing CLEC10A) as a result of SGRM therapy. Applicant further discloses methods, in a patient with cancer, using change from baseline in whole blood CLEC10A RNA as a biomarker to indicate increased numbers of a specific set of dendritic cells (e.g., dendritic cells expressing CLEC10A) in the patient, indicating that the patient is likely to have longer survival than other patients not showing indications of increased numbers of that specific set of dendritic cells. In embodiments, the cancer patient suffers from a cancer selected from ovarian, pancreatic, and prostate cancer.

The selective glucocorticoid receptor modulator (SGRM) may be a nonsteroidal compound comprising a heteroaryl ketone fused azadecalin structure. Compounds comprising a heteroaryl ketone fused azadecalin structure are described and disclosed in U.S. Pat. No. 8,859,774, which is hereby incorporated by reference in its entirety. In embodiments, the nonsteroidal SGRM is relacorilant, (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, having the formula:

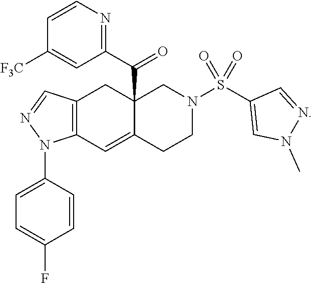 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

The SGRM may be a nonsteroidal compound comprising an octahydro fused azadecalin structure. Compounds comprising an octahydro fused azadecalin structure are described and disclosed in U.S. Pat. No. 10,047,082 which is hereby incorporated by reference in its entirety. In embodiments, the nonsteroidal SGRM is exicorilant, ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, having the formula:

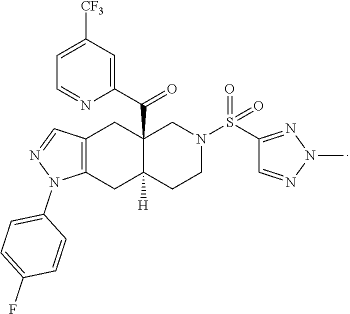 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

Accordingly, Applicant discloses herein a method of identifying a patient with cancer likely to benefit from administration of a SGRM and a cancer therapeutic, and treating said identified cancer patient, the method comprising: measuring a baseline level in a sample obtained from the cancer patient, of an RNA encoding a gene selected from CDKN1C, TNFRSF17, BRIP1, PDK1, CLEC10A, FPR3, CCR2, LILRB4, and CD86; administering a SGRM and a cancer therapeutic to said cancer patient; measuring a change in the level, as compared to said baseline level, in a sample obtained from the cancer patient following said administration of a SGRM and a cancer therapeutic, of said RNA encoding a gene selected from CDKN1C, TNFRSF17, BRIP1, PDK1, CLEC10A, FPR3, CCR2, LTLRB4, and CD86; wherein a change of said RNA level that is at least 40% as compared to the baseline level identifies the cancer patient as likely to benefit from further administration of said SGRM and said cancer therapeutic; and continuing said administration of the SGRM and the cancer therapeutic to said patient identified as likely to benefit from said treatment, thereby treating said identified cancer patient. In embodiments, the benefit to the identified cancer patient comprises increased survival.

Applicant further discloses herein a method of identifying a patient with cancer as having elevated cortisol activity, and treating said identified patient with cancer, the method comprising: measuring a baseline level, in a sample obtained from a patient with cancer, of an RNA encoding a gene selected from FKBP5, GSK3B, PIK3CG, MCL1, and CLEC10A; determining if said baseline level of an RNA encoding FKBP5, GSK3B, PIK3CG, or MCL1 is greater than a normal level of said RNA, wherein said normal level of the RNA is determined from the average level of the RNA in a cohort of at least 10 normal subjects, determining if said baseline level of an RNA encoding CLEC10A is lower than a normal level of said RNA encoding CLEC10A, wherein said normal level of an RNA encoding CLEC10A is determined from the average level of the RNA encoding CLEC10A in a cohort of at least 10 normal subjects, wherein if the baseline level of an RNA encoding FKBP5, GSK3B, PIK3CG, or MCL1 is greater than said normal level, the patient with cancer is identified as a patient with cancer having elevated cortisol activity, and wherein if the baseline level of an RNA encoding CLEC10A is lower than said normal level, the cancer patient is identified as a patient with cancer having elevated cortisol activity; then administering a selective glucocorticoid receptor modulator (SGRM) and a cancer therapeutic to said patient with cancer identified as having elevated cortisol activity, thereby treating the identified patient with cancer.

Applicant discloses herein yet a further method: a method of identifying an active dose of a SGRM in a patient with cancer, comprising: measuring a baseline level, in a sample obtained from the patient, of an RNA encoding a gene selected from CDKN1C, TNFRSF17, BRIP1, PDK1, CLEC10A, FPR3, CCR2, LILRB4, and CD86; administering a dose of a SGRM and of a cancer therapeutic to said cancer patient; then measuring, in a sample obtained from the patient, a change in the level, as compared to the baseline level, of an RNA encoding a gene selected from CDKN1C, TNFRSF17, BRIP1, PDK1, CLEC10A, FPR3, CCR2, LTLRB4, and CD86; wherein a dose of said SGRM that results in a) a decrease in the level of an RNA encoding CDKN1C, TNFRSF17, BRIP1, or PDK1 or b) an increase in the level of an RNA encoding CLEC10A, FPR3, CCR2, LTLRB4, and CD86 that is at least 40% as compared to the corresponding baseline level identifies the SGRM dose as an active dose. In embodiments, more than one SGRM dose, each SGRM dose having a different amount of SGRM than the other(s), is administered to the patient in order to identify an active SGRM dose. In embodiments, the more than one SGRM dose comprises an initial SGRM dose having a first amount of SGRM, and one or more subsequent SGRM doses having amounts of SGRM greater than the first amount of SGRM. In embodiments, the more than one SGRM dose comprises a) an initial SGRM dose having a first amount of SGRM, and b) one or more subsequent SGRM doses having amounts of SGRM lesser than the first amount of SGRM. In embodiments, the more than one SGRM dose comprises at least three SGRM doses, comprising a) an initial SGRM dose having a first amount of SGRM; b) a subsequent SGRM dose having an SGRM amount different than the first amount of SGRM; and c) a third SGRM dose between the other SGRM doses (having an amount of SGRM that is an amount that is greater than one of the other SGRM doses and that is less than the other SGRM dose).

Applicant discloses herein methods of increasing, in a patient with cancer, RNA levels encoding one or more of CLEC10A, FPR3, CCR2, LTLRB4, and CD86 comprising administering an effective dose of a SGRM to the patient, or comprising administering an effective dose of a SGRM and a cancer therapeutic to the patient. Applicant discloses herein methods of decreasing, in a patient with cancer, RNA levels encoding one or more of CDKN1C, TNFRSF17, BRIP1, and PDK1 comprising administering an effective dose of a SGRM to the patient, or comprising administering an effective dose of a SGRM and a cancer therapeutic to the patient. In embodiments, the SGRM inhibits GR activation. In embodiments, the patient with cancer suffers from a cancer selected from ovarian, pancreatic, and prostate cancer. It is believed that such alterations in RNA levels may provide benefit to patients with cancer. For example, among those patients with cancer treated with SGRM and a taxane, the patients with cancer that had increased CLEC10A experienced longer overall survival as compared to the patients with cancer receiving the same treatment but who did not have increased CLEC10A levels. In addition, patients with cancer receiving SGRM and a taxane and having an increase in CLEC10A had longer overall survival than those patients with cancer who were treated with taxane alone (regardless of CLEC10A change in the taxane-treated population.

Applicant discloses herein methods of detecting indications of increased numbers of dendritic cells (e.g., dendritic cells expressing CLEC10A); such increased numbers of such dendritic cells may be detected in a patient with cancer, and may indicate an enhanced immune response in the patient, and may indicate a beneficial effect of cancer treatment. Such increased numbers of dendritic cells (e.g., dendritic cells expressing CLEC10A) in the cancer patient receiving SGRM and a cancer therapeutic (e.g., a taxane or an antiandrogen) may indicate that the cancer patient is likely to have longer survival than other patients not showing such increased numbers of dendritic cells receiving the same SGRM and cancer therapeutic. In embodiments, the cancer patient suffers from a cancer selected from ovarian, pancreatic, and prostate cancer.

In embodiments of the methods disclosed herein directed to patients with cancer, the cancer patient may suffer from a cancer selected ovarian cancer, pancreatic cancer, and prostate cancer. In embodiments, the sample obtained from the cancer patient is a blood sample, e.g., a sample of whole blood obtained from the cancer patient. In embodiments, the cancer therapeutic is a taxane, or is an antiandrogen, or is another cancer therapeutic. In embodiments, the cancer therapeutic is a taxane selected from paclitaxel and nab-paclitaxel. In embodiments, the cancer therapeutic is the antiandrogen enzalutamide. In embodiments, the SGRM is a GR antagonist (GRA). In embodiments, the SGRM is relacorilant, or is exicorilant.

Applicant discloses herein yet another method: a method of identifying an active dose of a selective glucocorticoid receptor modulator (SGRM) in a patient with Cushing's syndrome, the method comprising: measuring a baseline level, in a sample obtained from said patient with Cushing's syndrome, of an RNA encoding a gene selected from CDKN1C, TNFRSF17, BRIP1, PDK1, CLEC10A, FPR3, CCR2, LILRB4, and CD86; administering a dose of a SGRM to the patient with Cushing's syndrome; then measuring, in a sample obtained from the patient with Cushing's syndrome, a change in the level, as compared to said baseline level, of an RNA encoding a gene selected from CDKN1C, TNFRSF17, BRIP1, PDK1, CLEC10A, FPR3, CCR2, LILRB4, and CD86, wherein a dose of said SGRM that results in a) a decrease in the level of RNA encoding CDKN1C, TNFRSF17, BRIP1, PDK1, or b) an increase in the level of RNA encoding CLEC10A, FPR3, CCR2, LILRB4, and CD86 that is at least 40% as compared to the baseline level is identified as an active dose of the SGRM. In embodiments, the method of identifying an active dose of a SGRM in a patient with Cushing's syndrome further comprises treating said patient with Cushing's syndrome, said treatment comprising: administering to the patient a further active dose of the SGRM, said further active dose consisting of the SGRM dose identified by the method, effective to treat the patient with Cushing's syndrome. In embodiments, more than one SGRM dose, each SGRM dose having a different amount of SGRM than the other(s), is administered to the patient in order to identify an active SGRM dose. In embodiments, the more than one SGRM dose comprises an initial SGRM dose having a first amount of SGRM, and one of more subsequent SGRM doses having amounts of SGRM greater than the first amount of SGRM. In embodiments, the more than one SGRM dose comprises a) an initial SGRM dose having a first amount of SGRM, and b) one or more subsequent SGRM doses having amounts of SGRM lesser than the first amount of SGRM. In embodiments, the more than one SGRM dose comprises at least three SGRM doses, comprising a) an initial SGRM dose having a first amount of SGRM; b) a subsequent SGRM dose having an SGRM amount different than the first amount of SGRM; and c) a third SGRM dose between the other SGRM doses. In embodiments, the sample obtained from the patient with Cushing's syndrome is a blood sample, e.g., a sample of whole blood obtained from the patient with Cushing's syndrome. In embodiments, the sample obtained from a patient with Cushing's syndrome is a saliva sample, or is a urine sample (e.g., a urine sample collected over a 24 hour period). In embodiments, the SGRM is relacorilant, or is exicorilant.

### B. Definitions

The term “about” denotes a range encompassing ±10% of the value to which it refers (e.g., “about 100” denotes a value between 90 and 110, inclusive).

As used herein, the terms “cancer therapeutic” and “chemotherapeutic agent”, and plurals and grammatical variants thereof, are used interchangeably, and refer to agents that have the property of killing cancer cells, or inhibiting cancer cell growth, or reducing cancer proliferation, or reducing metastasis, or are otherwise useful in the treatment of cancer. Examples of such chemotherapeutic agents include those disclosed in U.S. Pat. No. 10,568,880, hereby incorporated by reference in its entirety (see, e.g., columns 27-30). These agents include, but are not limited to antimicrotubule agents (e.g., taxanes and vinca alkaloids), topoisomerase inhibitors and antimetabolites (e.g., nucleoside analogs acting as such, for example, Gemcitabine), mitotic inhibitors, alkylating agents, antimetabolites, anti-tumor antibiotics, mitotic inhibitors, anthracyclines, intercalating agents, agents capable of interfering with a signal transduction pathway, agents that promote apoptosis, proteosome inhibitors, and alike.

As used herein, the term “taxane” refers to diterpene compounds useful as chemotherapeutic agents in cancer treatments. Exemplary taxanes include but are not limited to paclitaxel, nab-paclitaxel, cabazitaxel, and docetaxel. Nab-paclitaxel is nanoparticle albumin-bound paclitaxel (marketed as ABRAXANE© by Abraxis Bioscience). Other taxanes useful as chemotherapeutic agents in cancer treatments include docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

As used herein, the term “antiandrogen” refers to a therapeutic agent that reduces or blocks the action of androgens, or that reduces the production or levels of androgens in a subject. Antiandrogens are used, for example, in the treatment of prostate cancer. Androgen receptor antagonists are antiandrogens. Antiandrogens include, but are not limited to, enzalutamide (marketed as Xtandi©; also known as MDV-3100); cyproterone acetate; abiraterone acetate; spironolactone; flutamide; bicalutamide; darolutamide; nilutamide; goserelin; triptorelin; histrelin; and leuprolide. As used herein, the term “antiandrogen” also includes androgen deprivation therapy.

As used herein, “RNA” refers to ribonucleic acid, and may refer to an RNA that encodes for a particular protein (i.e., messenger RNA (mRNA)). The terms “mRNA” and “RNA” are used interchangeably herein.

Levels of RNA may be determined from samples obtained from a patient; for example, may be determined from a blood sample obtained from the patient. A blood simple may be a sample of whole blood, or of serum, or of plasma. For example, expression levels of several genes in patients receiving exicorilant were measured by NanoString techniques (NanoString Technologies, Inc., Seattle, WA).

As used herein, “nanostring”, “nanostring technique”, “nanostring technology”, and the like refer to techniques for, and the use of, amplification-free measurements of nucleic acid content of samples by counting target molecules directly. Nanostring techniques (Nanostring Technologies, Inc., Seattle, WA) may be used to measure the levels of multiple nucleic acid (RNA or DNA) targets in a sample. Sequence-specific probes that hybridize to their target nucleic acid sequences (e.g., RNA sequences) are used to detect the targets. These probes carry fluorescent molecules that allow detection and quantification of targets, including detection and quantification of each of multiple targets in a single sample.

Other methods for the detection of nucleic acid targets include, for example, Polymerase Chain Reaction (PCR) methods described, for example, in U.S. Pat. No. 4,683,195; and generally in Mullis et al., Cold Spring Harbor Symp. Quant. Biol. 51:263 (1987); Erlich, ed., PCR Technology (Stockton Press, N Y, 1989).

Methods of determining RNA levels include, without limitation, reverse transcription polymerase chain reaction (rt-PCR or RT-PCR). In rt-PCR, reverse transcriptase (RT) is used to make a complementary DNA (cDNA) from RNA, and the cDNA is then amplified by PCR to produce multiple copies of DNA to amplify the cDNA copies for detection and/or amplification of the target polynucleotide. RT-PCR requirements include a reverse transcriptase, a DNA polymerase (e.g., a thermostable DNA polymerase), deoxynucleotides (typically as deoxynucleotide tri-phosphates (“dNTPs”) such as dATP, dTTP, dGTP, and dCTP), and appropriate buffer solutions. See, e.g., U.S. Pat. Nos. 5,322,770 and 5,310,652, which are hereby incorporated by reference in their entirety.

As used herein, “real-time PCR” refers to PCR amplification methods in which the progress, or extent, of target amplification is monitored during the course of the assay (e.g., at each thermal cycle). Progress of the amplification reactions may be monitored, for example, detecting the amount of fluorescence or absorbance of reporter molecules. Suitable reporter molecules include intercalating dyes (which are detectable when bound to double-stranded DNA, or to the minor groove of DNA, such as ethidium bromide and SYBR Green dye); fluorogenic probes, such as self-quenching dyes, or dye pairs (the pairs including a dye and a quencher) attached to primers (which fluoresce when the primer is bound to target, but do not produce significant fluorescence when not hybridized to target nucleic acid molecules); and other reporter molecules.

As used herein, “rRT-PCR” refers to reverse-transcription real-time PCR. rRT-PCR is real-time PCR applied to RNA targets, using reverse-transcription PCR to amplify nucleic acids based on RNA target molecules, and monitoring the amplification using real-time PCR methods. As used herein, “rRT-PCR” refers to reverse-transcription real-time PCR. rRT-PCR is real-time PCR applied to RNA targets, using reverse-transcription PCR to amplify nucleic acids based on RNA target molecules, and monitoring the amplification using real-time PCR methods. Reverse-transcription PCR methods provide the DNA substrate required for PCR by contacting a sample, under the appropriate conditions, with a reverse transcriptase and producing cDNA copies of RNA molecules in the sample.

The gene names and accession IDs of many genes are listed in the table below, with some additional detail on the region of the gene that was explicitly measured by the labeled RNA probe. In the following Table, gene names are listed in lower case italic type. However, gene names, which also refer to RNA encoding those genes, are written in CAPITAL letters elsewhere in the present application.

TABLE 1

| Gene | Accession | Position | Target Sequence |
|---|---|---|---|
| cdkn1c | NM_000076.2 | 1606-1705 | TGTCCAGGCGTGGACCGCTCTGCCACGCACTAGCTCG |
| | | | GTTATTGGTTATGCCAAAGGCACTCTCCATCTCCCAC |
| | | | ATCTGGTTATTGACAAGTGTAACTTT (SEQ ID NO: 1) |
| tnfrsf17 | NM_001192.2 | 306-405 | TCTAATACTCCTCCTCTAACATGTCAGCGTTATTGTAA |
| | | | TGCAAGTGTGACCAATTCAGTGAAAGGAACGAATGC |
| | | | GATTCTCTGGACCTGTTTGGGACTGA (SEQ ID NO: 2) |
| brip1 | NM_032043.2 | 1571-1670 | TAGATAGTATGGTCAACAATAATATAAGGAAGAAAG |
| | | | ATCATGAACCCCTACGAGCTGTGTGCTGTAGCCTCAT |
| | | | TAATTGGTTAGAAGCAAACGCTGAATA (SEQ ID NO: 3) |
| pdk1 | NM_002610.3 | 402-501 | TTCTCAGGACACCATCCGTTCAATTGGTACAAAGCTG |
| | | | GTATATCCAGAGTCTTCAGGAGCTTCTTGATTTTAAG |
| | | | GACAAAAGTGCTGAGGATGCTAAAGC (SEQ ID NO: 4) |
| clec10a | NM_182906.2 | 431-530 | TGTCCCTGGGCCTCGGCCTCCTGCTGCTGGTCATCATC |
| | | | TGTGTGGTTGGATTCCAAAATTCCAAATTTCAGAGGG |
| | | | ACCTGGTGACCCTGAGAACAGATTT (SEQ ID NO: 5) |
| fpr3 | NM_002030.4 | 734-833 | TTTGCATTCTGGGGTGACACTGCTGTAGAGAGGTTGA |
| | | | ACGTGTTCATTACCATGGCCAAGGTCTTTCTGATCCTC |
| | | | CACTTCATTATTGGCTTCAGCGTGC (SEQ ID NO: 6) |
| ccr2 | NM_ | 439-538 | GAGAAAGTGGATTGAACAAGGACGCATTTCCCCAGT |
| | 001123041.2 | | ACATCCACAACATGCTGTCCACATCTCGTTCTCGGTTT |
| | | | ATCAGAAATACCAACGAGAGCGGTGA (SEQ ID NO: 7) |
| lilrb4 | NM_ | 592-691 | TTTCAGCCCTGCCGAGTCCTCTTGTGACCTCAGGAAA |
| | 001278426.3 | | GAGCGTGACCCTGCTGTGTCAGTCACGGAGCCCAATG |
| | | | GACACTTTTCTTCTGATCAAGGAGCG (SEQ ID NO: 8) |
| cd86 | NM_ | 1686-1785 | CATGAAATGTCTGGTCTGTCCACCCCATCAACAAGTC |
| | 001206924.1 | | TTGAAACAAGCAACAGATGGATAGTCTGTCCAAATG |
| | | | GACATAAGACAGACAGCAGTTTCCCTG (SEQ ID NO: 9) |
| fkbp5 | NM_ | 541-640 | ACCATTTGTCTTTAGTCTTGGCAAAGGCCAAGTCATC |
| | 001145775.1 | | AAGGCATGGGACATTGGGGTGGCTACCATGAAGAAA |
| | | | GGAGAGATATGCCATTTACTGTGCAAA (SEQ ID |
| | | | NO: 10) |
| pik3cg | NM_002649.2 | 2126-2225 | AAAACATACCAATTGTTGGCCAGAAGGGAAGTCTGG |
| | | | GATCAAAGTGCTTTGGATGTTGGGTTAACAATGCAGC |
| | | | TCCTGGACTGCAACTTCTCAGATGAAA (SEQ ID NO: 11) |
| mcl1 | NM_021960.3 | 1261-1360 | GCTGTAACTTCCTAGAGTTGCACCCTAGCAACCTAGC |
| | | | CAGAAAAGCAAGTGGCAAGAGGATTATGGCTAACAA |
| | | | GAATAAATACATGGGAAGAGTGCTCCC (SEQ ID |
| | | | NO: 12) |
| gsk3b | NM_002093.2 | 926-1025 | ACTGATTATACCTCTAGTATAGATGTATGGTCTGCTG |
| | | | GCTGTGTGTTGGCTGAGCTGTTACTAGGACAACCAAT |
| | | | ATTTCCAGGGGATAGTGGTGTGGATC(SEQ ID NO: 13) |

As used herein, “subject”, “normal subject” and “control subject”, including plural and grammatical variants thereof, refer to healthy subjects, that is, subjects who do not suffer from a disease or disorder; e.g., who do not suffer from cancer, or do not suffer from Cushing's syndrome.

“Patient”, “patient in need”, and the like refer to a person having, or suspected of having, a disease or condition which may be treated by administration of a therapeutic drug or combination of drugs.

As used herein, the terms “Cushing's syndrome”, and “Cushing's” refer to the condition caused by the excessive production of the glucocorticoid cortisol by the adrenal cortex or by an ectopic (non-adrenal) source, such as a tumor. The term “Cushing's syndrome” includes endogenous Cushing's syndrome and ectopic Cushing's syndrome. The condition is often due to the presence of a tumor or hyperplasia that exhibits unregulated secretion of adrenocorticotropic hormone (ACTH), or of cortisol itself. Cushing's syndrome presents some or all of an array of symptoms caused by excess cortisol. Such symptoms include, for example, elevated blood pressure, elevated blood glucose, increased weight (typically in the mid-section, and in the face causing a characteristic “moon-face”), immune suppression, thin skin, acne, depression, hirsutism, and other symptoms. Patients with Cushing's syndrome include patients with Cushing's Disease, and include those suffering subclinical Cushing's syndrome and difficult-to-diagnose Cushing's syndrome.

The terms “Cushing Disease” and “Cushing's Disease” refer to pituitary-dependent Cushing's syndrome, e.g., excess cortisol caused by pituitary abnormality (typically a pituitary tumor), e.g., conditions in which the pituitary gland releases too much ACTH as a result of a tumor located in or near the pituitary gland, or as a result of excess growth (hyperplasia) of the pituitary gland. Cushing Disease is a form of Cushing's syndrome.

The term “endogenous Cushing's syndrome” refers to a form of Cushing's syndrome, where the excess cortisol level is caused by the body's own overproduction of cortisol.

As used herein, a “patient suffering from Cushing's syndrome” refers to any patient suffering from Cushing's syndrome, including endogenous Cushing's syndrome; Cushing's Disease; or a condition associated with Cushing's syndrome. A condition associated with Cushing's syndrome may be, without limitation, hypercortisolism; hyperglycemia secondary to hypercortisolism; type 2 diabetes mellitus or glucose intolerance; such a condition in a patient with endogenous Cushing's syndrome who has failed surgery; such a condition in a patient with endogenous Cushing's syndrome, who is not a candidate for surgery; and other conditions associated with Cushing's syndrome.

“Standard control” as used herein refers to a sample comprising a predetermined amount of an analyte (such as an RNA of interest or cortisol) suitable for the use of an application of the present invention, in order to serve as a comparison basis for providing an indication of the relative amount of the analyte that is present in a test sample. A sample serving as a standard control provides an average amount of an analyte such as cortisol that is representative for a defined sample type (e.g., plasma, serum, saliva, or urine) taken at a defined time of the day (e.g., 8 AM) from an average individual.

The term “measuring the level,” in the context of cortisol, an RNA encoding a gene, or other analyte, refers to determining, detecting, or quantitating the amount, level, or concentration of the analyte in a sample obtained from a subject.

As used herein, a “sample” may be any fluid or tissue obtained from a patient, including, e.g., a blood sample, a urine sample, a saliva sample, or other sample.

As used herein, a “blood sample” may be a whole blood sample, serum sample, plasma sample, or blood cell sample as appropriate for measuring an analyte level by art-known methods according to conventional use. Similarly, “blood level” of a particular analyte maybe the level of the analyte in the whole blood, serum, plasma, or blood cells. For example, the blood level of cortisol, or of an RNA encoding a gene, or other analyte, maybe the level of the analyte in a whole blood, serum, or plasma sample taken from a subject being tested.

The term “morning serum sample” refers to a serum sample obtained from a human subject in the morning, where morning may be a time between about 6 AM to about 12 noon, or between about 7 AM and about 11 AM, or other time understood as during the morning.

The term “morning serum cortisol sample” refers to a morning serum sample in which the level, e.g., the concentration, of cortisol is measured.

The term “cortisol” refers to the naturally occurring glucocorticoid hormone (also known as hydrocortisone) that is produced by the zona fasciculata of the adrenal gland. Cortisol has the structure:

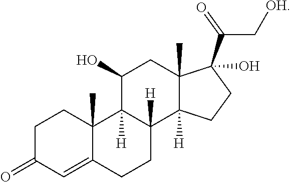

The term “total cortisol” refers to cortisol that is bound to cortisol-binding globulin (CBG or transcortin) and free cortisol (cortisol that is not bound to CBG). The term “free cortisol” refers to cortisol that is not bound to cortisol-binding globulin (CBG or transcortin). As used herein, the term “cortisol” refers to total cortisol, free cortisol, and/or cortisol bound to CBG.

Cortisol levels may be determined, e.g., by measuring cortisol in blood (e.g., serum or plasma), urine, saliva, tears, or other bodily fluid. In embodiments, cortisol levels may be measured in serum samples obtained in the morning (morning serum cortisol). Plasma samples may be used in a similar fashion to assess cortisol level in a subject.

The level of cortisol can be measured in a sample (of, e.g., whole blood, serum, plasma, saliva, urine, tears, or other biological fluid) using various methods, including but not limited to, immunoassays, e.g., competitive immunoassay, radioimmunoassay (MA), immunofluorometric enzyme assay, and ELISA; competitive protein-binding assays; liquid chromatography (e.g., HPLC); and mass spectrometry, e.g., high-performance liquid chromatography/triple quadrupole-mass spectrometry (LC-MS/MS). In preferred embodiments, cortisol levels are measured using LC-MS/MS, such as performed by Quest Diagnostics (Secaucus, N.J. 07094).

As used herein, the terms “baseline” and “baseline level”, including plural and grammatical variants thereof, refer to the level of an analyte measured in a sample obtained from a subject or from a patient prior to the subject or patient receiving a treatment (e.g., prior to administration of a SGRM or cancer therapeutic to the subject or patient).

As used herein, “normal level” and “control level”, including plural and grammatical variants thereof, refer to the average level of an analyte as determined by measurements of samples obtained from multiple normal subjects. For comparison, the same types of measurements (e.g., plasma or serum; salivary; or urinary) must be the compared. A normal, or control, level of an analyte may be measured in a sample, or group of samples, obtained from a healthy subject or a group of healthy subjects in the absence of administration of a drug or of receiving a treatment. For example, a normal level of an analyte such as an mRNA encoding a gene of interest may be determined by obtaining blood samples from 10 or more healthy subjects, measuring the level of the analyte in each of the samples, and calculating an average of the measured analyte levels from these sample measurements; that average provides a level that is termed a “normal level” or a “control level” of that analyte.

The term “normal cortisol level” refers to the average level of cortisol as determined by measurements of samples (e.g., serum samples) obtained from multiple normal subjects. A normal cortisol level may be known to, or may be ascertainable by, those of ordinary skill in the art. For example, as reported by Putignano et al. in a study of plasma cortisol in healthy women (European Journal of Endocrinology 145:165-171 (2001)), normal plasma cortisol was about 420 nanomoles per liter (nmol/l) at 8 AM (morning); about 250 nmol/l at 5 PM (evening); and about 90 nmol/l at 12 PM (late night). Salivary cortisol measurements were about 14 nmol/l at 8 AM (morning); about 7 nmol/l at 5 PM (evening); and about 5 nmol/l at 12 PM (late night). Urinary free cortisol levels were about 130 nmol per 24 hours (nmol/24 h). Cortisol levels are suppressed by the dexamethasone suppression test (DST), as indicated by the plasma cortisol level of about 24 nmol/l following DST and the salivary cortisol level of about 4 nmol/l following DST. Cortisol levels in healthy men are believed to be similar to those reported by Putignano et al.

The terms “excess”, “excess level”, “elevated level”, “elevated amount”, or “elevated concentration” refers to a level or amount of the analyte that is higher than the normal or baseline value for that analyte, and particularly refer to levels that are significantly higher than normal or baseline.

As used herein, the terms “excess cortisol activity”, “elevated cortisol activity”, “elevated cortisol”, “elevated cortisol level”, “cortisol excess”, and the like refer to cortisol levels, however measured, that are greater than about 1.5 times, or greater than about 2 times, normal cortisol levels. The median urinary free cortisol (UFC) level was measured as 17 μg/day (47 nmol/day) in a study of patients suffering from castration-resistant prostate cancer. Thus, for example, in studies disclosed in the present application, patients having UFC greater than 17 g/day (47 nmol/day) were considered to have elevated cortisol activity.

The term “steroidal backbone” in the context of glucocorticoid receptor antagonists containing such refers to glucocorticoid receptor antagonists that contain modifications of the basic structure of cortisol, an endogenous steroidal glucocorticoid receptor ligand. The basic structure of a steroidal backbone is illustrated below:

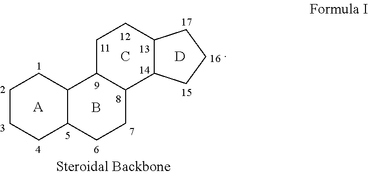

As used herein, the phrase “nonsteroidal backbone” in the context of SGRMs refers to SGRMs that do not share structural homology to, or are not modifications of, cortisol with its steroid backbone containing seventeen carbon atoms, bonded in four fused rings.

The term “glucocorticosteroid” (“GC”) or “glucocorticoid” refers to a steroid hormone that binds to a glucocorticoid receptor. Glucocorticosteroids are typically characterized by having 21 carbon atoms, an α,β-unsaturated ketone in ring A, and an α-hydroxy group attached to ring D. They differ in the extent of oxygenation or hydroxylation at C-11, C-17, and C-19; see Rawn, “Biosynthesis and Transport of Membrane Lipids and Formation of Cholesterol Derivatives,” in Biochemistry, Daisy et al. (eds.), 1989, pg. 567.

As used herein, the term “glucocorticoid receptor” (“GR”) refers to the type II GR, a family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “selective glucocorticoid receptor modulator” (SGRM) refers to any composition or compound which modulates GC binding to GR, or modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor modulator bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor modulator binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor modulator binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. Relacorilant is a SGRM.

“Glucocorticoid receptor antagonist” (GRA) refers to any compound which inhibits GC binding to GR, or which inhibits any biological response associated with the binding of GR to an agonist. Accordingly, GR antagonists can be identified by measuring the ability of a compound to inhibit the effect of dexamethasone. TAT activity can be measured as outlined in the literature by A. Ali et al., J. Med. Chem., 2004, 47, 2441-2452. A GRA is a compound with an IC₅₀ (half maximal inhibition concentration) of less than 10 micromolar. See Example 1 of U.S. Pat. No. 8,859,774, the entire contents of which is hereby incorporated by reference in its entirety.

As used herein, the term “selective glucocorticoid receptor antagonist” (SGRA) refers to any composition or compound which inhibits GC binding to GR, or which inhibits any biological response associated with the binding of a GR to an agonist (where inhibition is determined with respect to the response in the absence of the compound). By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor antagonist bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor antagonist binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor antagonist binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. Relacorilant is a SGRA.

Nonsteroidal GRA, SGRA, GRM, and SGRM compounds include compounds comprising a fused azadecalin structure (which may also be termed a fused azadecalin backbone), compounds comprising a heteroaryl ketone fused azadecalin structure (which may also be termed a heteroaryl ketone fused azadecalin backbone), compounds comprising an octahydro fused azadecalin structure (which may also be termed an octahydro fused azadecalin backbone). Exemplary nonsteroidal GRA, SGRA, GRM, and SGRM compounds comprising a fused azadecalin structure include those described in U.S. Pat. Nos. 7,928,237 and 8,461,172. Exemplary nonsteroidal GRA, SGRA, GRM, and SGRM compounds comprising a heteroaryl ketone fused azadecalin structure include those described in U.S. Pat. No. 8,859,774. Exemplary nonsteroidal GRA, SGRA, GRM, and SGRM compounds comprising an octahydro fused azadecalin structure include those described in U.S. Pat. No. 10,047,082.

Exemplary heteroaryl-ketone fused azadecalin compounds are described in U.S. Pat. No. 8,859,774; in U.S. Pat. No. 9,273,047; in U.S. Pat. No. 9,707,223; and in U.S. Pat. No. 9,956,216, all of which patents are hereby incorporated by reference in their entireties. In embodiments, the SGRM is a heteroaryl-ketone fused azadecalin. In embodiments, the SGRA is the compound (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone (Example 18 of U.S. Pat. No. 8,859,774), also known as “relacorilant” and as “CORT125134”, which has the following structure:

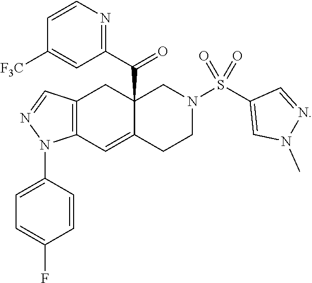 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

In embodiments, the heteroaryl-ketone fused azadecalin SGRM is the compound (R)-(1-(4-fluorophenyl)-6-((4-(trifluoromethyl)phenyl)sulfonyl)-4,4a,5,6,-7,8-hexahydro-1H-pyrazolo[3,4-g]isoquinolin-4a-yl)(thiazol-2-yl)methanone (termed “CORT122928”), which has the following structure:

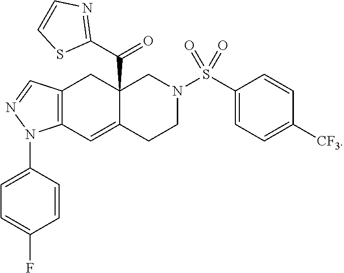 [SMILES: O=C(c1nccs1)[C@]12Cc3cnn(-c4ccc(F)cc4)c3C=C1CCN(S(=O)(=O)c1ccc(C(F)(F)F)cc1)C2]

In embodiments, the heteroaryl-ketone fused azadecalin SGRM is the compound (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone (dazucorilant; also known as “CORT113176”), which has the following structure:

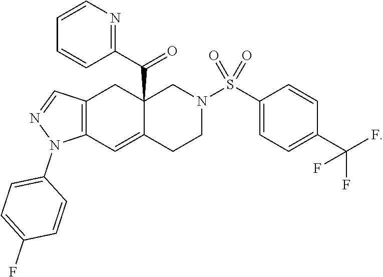 [SMILES: O=C(c1ccccn1)[C@]12Cc3cnn(-c4ccc(F)cc4)c3C=C1CCN(S(=O)(=O)c1ccc(C(F)(F)F)cc1)C2]

In embodiments, the SGRM is an octahydro fused azadecalin. In embodiments, the octahydro fused azadecalin is exicorilant (also known as CORT125281), ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, which the octahydro fused azadecalin has the formula:

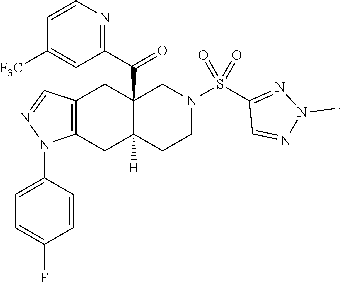 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

As used herein, the terms “active amount”, active dose”, “effective amount” and the like refer to an amount of a pharmacological agent that is effective to elicit a response in the subject or patient to whom the agent has been administered. Such a response may be, for example, a change in the level of an analyte (e.g., a RNA of interest, or a cortisol level) in samples obtained from the subject or patient. Such a change, may be, e.g., an at least 40% change in analyte level. An active dose thus will elicit a response in a subject or patient to whom that dose is administered. An active dose may be effective to treat, eliminate, or mitigate at least one symptom of the disease being treated. In some cases, an “effective amount” can refer to an amount of an agent or of a pharmaceutical composition useful for exhibiting a detectable therapeutic or inhibitory effect. The effect can be detected by any assay method known in the art.

As used herein, the terms “administer,” “administering,” “administered,” “administration,” and the like, refer to providing a compound or a composition (e.g., one described herein), to a subject or patient. Thus, for example, “administration to a patient” refers to the delivery of a drug or other therapeutic into the body of a patient in need of treatment by the drug or therapeutic, effective to achieve a therapeutic effect. Administration may be by any suitable route of administration, including, for example, oral administration; intravenous administration; subcutaneous administration; parenteral administration; intra-arterial administration; nasal administration; topical administration; and other routes of administration.

“Treat”, “treating” and “treatment” refer to any indicia of success in the treatment or amelioration of a pathology or condition, including any objective or subjective parameter such as abatement; remission; diminishing of symptoms or making the pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters; including the results of a physical examination; histopathological examination (e.g., analysis of biopsied tissue); laboratory analysis of urine, saliva, tissue samples, serum, plasma, or blood; or imaging.

As used herein, the term “combination therapy” refers to the administration of at least two pharmaceutical agents to a subject to treat a disease. The two agents may be administered simultaneously, or sequentially in any order during the entire or portions of the treatment period. The at least two agents may be administered following the same or different dosing regimens. In some cases, one agent is administered following a scheduled regimen while the other agent is administered intermittently. For example, in some embodiments, an SGRM is administered daily; in other embodiments, a SGRM is administered intermittently, e.g., every two, three, or four days, or at other intervals. In some cases, both agents are administered intermittently. In some embodiments, the one pharmaceutical agent, e.g., a SGRM, is administered daily, and the other pharmaceutical agent, e.g., a chemotherapeutic agent, is administered every two, three, or four days, or at other intervals. In some embodiments, a chemotherapeutic agent is administered daily, and the other pharmaceutical agent, e.g., a SGRM, is administered every two, three, or four days, or at other intervals.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. Salts and solvates (in which the molecular moiety of defined chemical structure (“compound”) is associated with a molecule(s) of a solvent) are examples of such associated forms. A hydrate is a solvate in which the associated solvent is water. The recitation of a “compound” refers to the molecular moiety itself (of the recited structure), regardless of whether it exists in a free form or an associated form.

As used herein, the term “composition” is intended to encompass a product comprising the specified ingredients such as a compound disclosed herein, and tautomeric forms, derivatives, analogues, stereoisomers, polymorphs, deuterated species, pharmaceutically acceptable salts, esters, ethers, metabolites, mixtures of isomers, pharmaceutically acceptable solvates thereof, and pharmaceutically acceptable compositions in specified amounts, as well as any product which results, directly or indirectly, from combination of the specified ingredients in the specified amounts. The pharmaceutical compositions discussed herein are meant to encompass any composition made by admixing compounds discussed and their pharmaceutically acceptable carriers.

As used herein, the terms “pharmaceutically-acceptable excipient” and “pharmaceutically acceptable carrier” are intended to include any and all solvents, surfactants, dispersion media, coatings, antibacterial and antifungal agents, isotonic and absorption delaying agents, and the like, compatible with pharmaceutical administration. These terms refer to any substance that aids the administration of an active agent to—and absorption by—a subject and can be included in pharmaceutical compositions without causing a significant adverse toxicological effect on the patient. Except insofar as any conventional media or agent is incompatible with the active compound, use thereof in pharmaceutical compositions is contemplated. Non-limiting examples of pharmaceutically-acceptable excipients include water, NaCl, normal saline solutions, lactated Ringer's, normal sucrose, normal glucose, binders, fillers, disintegrants, encapsulating agents, plasticizers, lubricants, coatings, sweeteners, flavors and colors, and the like. One of ordinary skill in the art will recognize that other pharmaceutical excipients may be useful as well.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR modulator and disease or condition treated.

SGRMs can be used in combination with other active agents known to be useful in modulating a glucocorticoid receptor, or with adjunctive agents that may not be effective alone, but may contribute to the efficacy of the active agent.

In some embodiments, co-administration includes administering a SGRM within 0.5, 1, 2, 4, 6, 8, 10, 12, 16, 20, or 24 hours of a second active agent (e.g., a cancer therapeutic). Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a SGRM as discussed herein has been formulated in an acceptable carrier, it can be placed in an appropriate container and labeled for treatment of an indicated condition. For administration of a SGRM, such labeling would include, e.g., instructions concerning the amount, frequency and method of administration.

The pharmaceutical compositions can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

### I. Combination Therapies

Various combinations with a GRM or SGRM and another agent (or a combination of such agents and compounds) may be employed to treat Cushing's syndrome, Cushing's Disease, or a cancer in the patient. By “combination therapy” or “in combination with”, it is not intended to imply that the therapeutic agents must be administered at the same time and/or formulated for delivery together, although these methods of delivery are within the scope described herein. The GRM or SGRM and the chemotherapeutic agent can be administered following the same or different dosing regimen. In some embodiments, the GRM or SGRM and the chemotherapeutic agent is administered sequentially in any order during the entire or portions of the treatment period. In some embodiments, the GRM or SGRM and the anticancer agent is administered simultaneously or approximately simultaneously (e.g., within about 1, 5, 10, 15, 20, or 30 minutes of each other). Non-limiting examples of combination therapies are as follows, with administration of the GRM or SGRM and the chemo agent for example, GRM or SGRM is “A” and the anticancer agent or compound, given as part of an chemo therapy regime, is “B”:

A/B/AB/A/BB/B/AA/A/BA/B/BB/A/AA/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic compounds or agents to a patient will follow general protocols for the administration of such compounds, taking into account the toxicity, if any, of the therapy. Surgical intervention may also be applied in combination with the described therapy.

### Examples

The following examples are provided by way of illustration only and not by way of limitation. Those of skill will readily recognize a variety of noncritical parameters which could be changed or modified to yield essentially similar results.

### Example 1. Assessing Glucocorticoid Receptor Modulation

### Ovarian Cancer Studies

The methods and results disclosed herein were obtained from studies including a randomized Phase 2 study of relacorilant plus nab-paclitaxel (rela+nab-pac) in patients with ovarian cancer (clinical trial NCT03776812).

### Methods

### Study Design

This phase 2, open-label, randomized, 3-arm study was performed in accordance with the principles of the Declaration of Helsinki, the Good Clinical Practice guidelines of the International Council on Harmonisation, and local regulatory requirements. The protocol was approved by the institutional review board or independent ethics committee at each investigative site. All patients or their legally authorized representatives provided written informed consent.

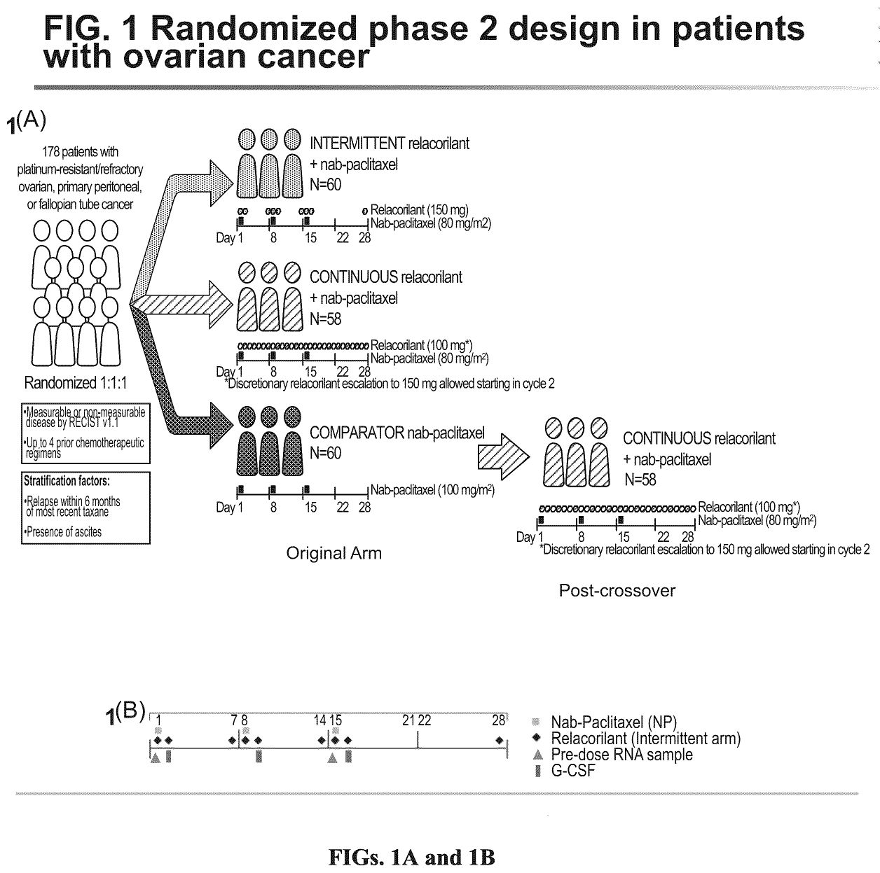

Patients were randomized 1:1:1 to one of the 3 treatment arms: a) nab-paclitaxel (80 mg/m²)+intermittent relacorilant (150 mg; administered orally on the day before [excluding cycle 1 day −1], the day of, and the day after nab-paclitaxel; “intermittent arm”); b) nab-paclitaxel (80 mg/m²)+continuous relacorilant (100 mg, administered orally once daily; “continuous arm”); and c) nab-paclitaxel monotherapy (100 mg/m²; “nab-paclitaxel-only arm”). (As used herein, “+” indicates “with”.) Across all arms, nab-paclitaxel was administered on days 1, 8, and 15 of each 28-day cycle. Study arms were stratified by treatment-free interval from most recent taxane (relapse within 6 months vs >6 months, i.e., patients experiencing relapse within 6 months of their most recent taxane treatment were placed in different arms than were patients experiencing relapse at times greater than 6 months after their most recent taxane treatment), and by presence of ascites (yes/no). Prophylactic G-CSF was mandated on the relacorilant+nab-paclitaxel arms and optional on the nab-paclitaxel alone arm. Patients (27 in total) that progressed on the nab-paclitaxel monotherapy arm were allowed to crossover to the nab-paclitaxel+continuous relacorilant arm (FIG. 1).

### Patient Population

Female patients (≥18 years old) with a histologic diagnosis of recurrent high-grade serous or endometrioid epithelial ovarian, primary peritoneal, or fallopian tube cancer or ovarian carcinosarcoma, for whom nab-paclitaxel was an appropriate treatment in the opinion of the investigator, were eligible to participate. Clear cell, mucinous, and borderline histologic subtypes were excluded. At least 1 prior line of platinum-based chemotherapy with platinum-free interval≤6 months or disease progression during or immediately after platinum-based therapy was required. Patients with primary platinum-resistant or primary platinum-refractory disease were also eligible to participate. Measurable or non-measurable disease by RECIST v1.1 and ≤4 prior chemotherapeutic or myelosuppressive lines (not including maintenance therapy) were allowed. Eastern Cooperative Oncology Group (ECOG) status 0 or 1 and adequate organ and bone marrow function were required.

### Assessments

Radiographic tumor assessments (computerized tomography with contrast or magnetic resonance imaging) were performed within 28 days prior to and every 8 weeks (±7 days) from cycle 1 day 1 until disease progression, including in patients who prematurely discontinued therapy. Tumor response was assessed by the investigator or local radiologist using RECIST v1.1.

### Specimen Collection

Blood was collected at cycle 1 day 1, prior to administration of relacorilant or nab-paclitaxel. Blood was also drawn in the morning, pre-dose, on cycle 1, day 15. Patients that progressed on the nab-paclitaxel monotherapy arm and then crossed over to the nab-paclitaxel+continuous relacorilant arm had collections at cycle 1 day 1 and cycle 1 day 15 on both arms. Blood (2.5 mL) was drawn into a PAXGene blood RNA tube (Qiagen) using a butterfly needle. The tube was sealed and gently inverted 10 times. The tube was frozen in dry ice and stored at −80° C. until RNA extraction.

### Pancreatic Cancer Studies

Open-Label Single-Arm Study of Relacorilant+Nab-Paclitaxel in Patients with Metastatic Pancreatic Ductal Adenocarcinoma (mPDAC) (NCT04329949)

This was as multicenter, open-label, single-arm study to assess the safety and efficacy of relacorilant in combination with nab-paclitaxel in patients with mPDAC. Patients with mPDAC were administered relacorilant and nab-paclitaxel, starting on Cycle 1 Day 1 and continuing until disease progression, unacceptable toxicity, or other treatment discontinuation criteria were met. Relacorilant (starting dose 100 mg, titrated in increments of 25 mg per cycle to a potential maximum dose of 150 mg) was taken once daily and nab-paclitaxel 80 mg/m² was administered on Days 1, 8, and 15 of each 28-day cycle.

### Patient Population

This study included male and female patients (≥18 years of age) with histologically confirmed mPDAC who had received at least 2 prior lines of therapy for PDAC, including at least 1 prior gemcitabine-based therapy and 1 prior fluoropyrimidine-based therapy. Patients must not have received more than 4 prior lines of cytotoxic or myelosuppressive therapy for PDAC and must have had a measurable lesion at Baseline per Response Evaluation Criteria for Solid Tumors, v1.1 (RECIST v1.1).

### Specimen Collection

Blood was collected at cycle 1 day 1, prior to administration of relacorilant or nab-paclitaxel in PAXgene RNA tubes. Blood was also drawn in the morning, pre-dose, on cycle 1 day 15.

### Prostate Cancer Studies

### Study of Exicorilant+Enzalutamide in Metastatic Castration Resistant Prostate Cancer (NCT03437941)

This was a Phase 1/2a study assessing the safety, tolerability, and PK of exicorilant in combination with enzalutamide in patients with metastatic castration-resistant prostate cancer (mCRPC). Study objectives were to identify the pharmacologically active dose and exposure range, characterize the PK of exicorilant in combination with enzalutamide, assess the potential for drug-drug interactions, and select appropriate doses of exicorilant+enzalutamide for further development.

Dose determination was conducted in 2 Segments. In Segment 1, mCRPC patients with progressive disease defined by PSA or imaging were enrolled into 3 sequential dose-finding cohorts to evaluate exicorilant twice daily under fasting conditions in combination with enzalutamide 160 mg daily (with or without enzalutamide lead-in). Segment 2 was randomized and double blinded for dose titration with respect to exicorilant in combination with enzalutamide (80 mg to 160 mg daily). All patients received a starting dose of 240 mg exicorilant once daily under fed conditions. Patients in Segment 2 were required to be on a stable dose of enzalutamide with a rising PSA and were randomized 3:1 to receive their established dose of enzalutamide+exicorilant as two dosing regimens: 240 mg exicorilant once daily with dose titration up to 320 mg once daily in 40 mg increments, or 240 mg exicorilant once daily and placebo (without increase of the active dose).

### Specimen Collection

Blood was collected at cycle 1 day 1, prior to administration of exicorilant or enzalutamide in PAXgene RNA tubes. Blood was also drawn in the morning, pre-dose, on cycle 1, day 15. Urine was collected for 24 consecutive hours the day before the cycle 1 day 1 visit.

### Study of Relacorilant+Enzalutamide in Metastatic CRPC Cancer (NCT03674814)

This was a Phase 1 study assessing the safety, tolerability, and PK of relacorilant in combination with enzalutamide in patients with mCRPC. The study objectives were to identify the pharmacologically active dose and select appropriate doses of relacorilant+enzalutamide for further development.

### Specimen Collection

Blood was collected at cycle 1 day 1, prior to administration of relacorilant or enzalutamide. Blood was also drawn in the morning, pre-dose, on cycle 1, day 15 in PAXgene RNA tubes.

Study of Prednisone in Healthy Volunteers (from Exicorilant SAD/MAD Study NCT03335956)

This was a Phase 1 single-ascending dose (SAD) and multiple-ascending dose (MAD) study to assess the safety and pharmacological activity of exicorilant. Co-dosing of exicorilant with prednisone 25 mg, and comparison to the effects of prednisone alone, established the pharmacological activity of exicorilant. Only the specimens collected after dosing with prednisone alone were analyzed for the studies described below.

### Specimen Collection

Blood was collected in the morning prior to dosing in PAXgene RNA tubes. 4 hours after a dose of prednisone 25 mg, blood was drawn again.

### Biomarker Analyses

### RNA Isolation and Quantification

Paired baseline and post-dose specimens were thawed and processed in the same batch. RNA was isolated using the PAXgene Blood RNA kit (Qiagen) using the protocol recommended by the manufacturer. RNA yield was quantified using a NanoDrop ND-2000 spectrophotometer (ThermoFisher Scientific). RNA was assessed using a custom 444-gene panel after mRNA sample preparation and hybridization using NanoString nCounter XT Assay, following the operation and maintenance instructions for the NanoString preparation station and digital analyzer. Specific RNA transcripts were quantified using a Nanostring nCounter FLEX instrument (NanoString Technologies) and analyzed using nSolver 4.0. (Nanostring Technologies, Seattle WA, USA).

### Data Normalization and Analysis

Housekeeping gene pairwise correlations were determined using nSolver 4.0 (NanoString Technologies). Test genes were normalized to the housekeeping genes HPRT1, PPIB, TRAP1, EEF1A1, and TBP. Water blank samples were used to determine the background signal for each probe. A reference standard (Agilent, cat #750500) were used to ensure consistent batch-to-batch assay performance. Fold change from baseline was calculated using the RNA counts at baseline and post-dose as follows:

Fold Change=log₂((post-treatment)/(baseline))

The log₂ change from baseline was calculated prior to assessing correlation coefficients or statistical significance. For baseline only analyses, gene abundance is reported as normalized counts.

Data were analyzed and adjusted P-values (with Benjamini-Yekutieli false discovery rate corrections) were determined in nSolver 4.0 (NanoString Technologies, Seattle, WA). Cross validated random forest techniques were used to derive a gene signature that could distinguish the relacorilant+nab-paclitaxel group from nab-paclitaxel alone (Ardigen SA, Krakow, Poland, Study WO5). Genes were ranked by p-value to determine those with a fold change that was most different between the nab-paclitaxel alone arm than the relacorilant+nab-paclitaxel arms. The resulting top 4 relacorilant-suppressed genes and top 4 relacorilant-induced genes were included in the 8-gene set used for further analysis.

### Bioinformatic Analyses

CLEC10A RNA levels associated with overall survival in ovarian cancer were determined using KM Plotter [Nagy et al., Scientific Reports, 11, 6047 (2021)] without any filtering (i.e., histology, stage, grade, CA-125, or treatment). Cell type expression data were collected from the EBI expression atlas [Papatheodorou et al., Nucleic Acids Research, 46, D246-D251 (2018)] and normalized. Normalization was conducted by determining the total counts for a single gene across all cell types, then calculating the counts for each cell type as a fraction of the total counts.

### 24 hr Urinary Free Cortisol Quantification

Cortisol was extracted from urine in 2-Butanol:Ethyl Acetate:Hexane (25.0:300:675, v/v/v), dried and reconstituted in Water:Methanol (500:500, v/v), and quantified by LC-MS/MS. Analytes were separated using a Kinetex Biphenyl (Phenomenex) column in a gradient of Water:Propionic Acid:1.25% Citric Acid (800:2.40:0.173, v/v/v) and Methanol:Acetonitrile:Propionic Acid:1.25% Citric Acid Monohydrate (400:˜400:2.40:0.173, v/v/v/v). HPLC (1200 Series binary SL, Agilent) was coupled to a tandem quadrupole mass spectrometer (Triple quad 5500, AB Sciex).

### Results

Discovery of Genes Modified by SGRM in Patients with Ovarian Cancer

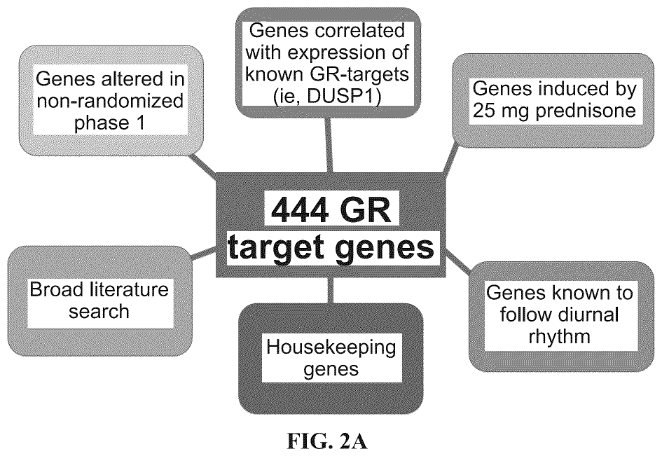

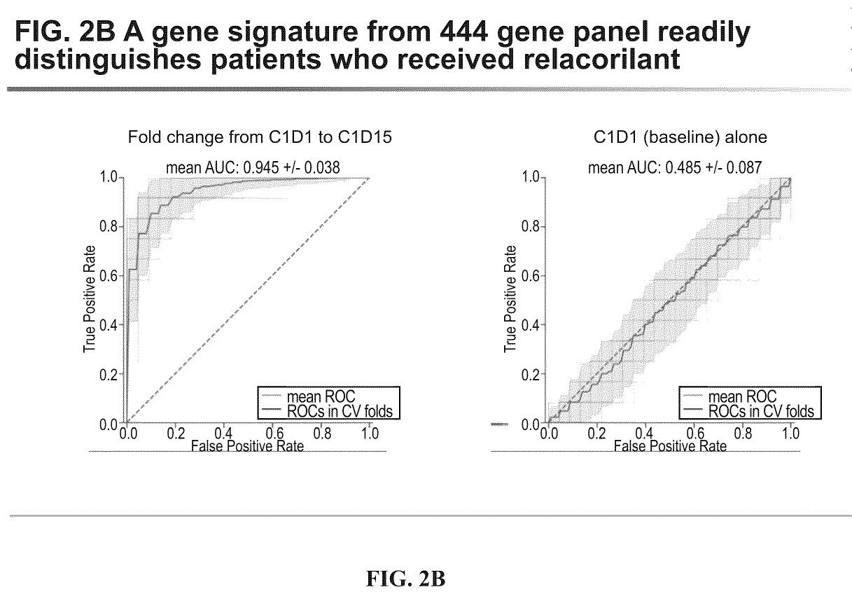

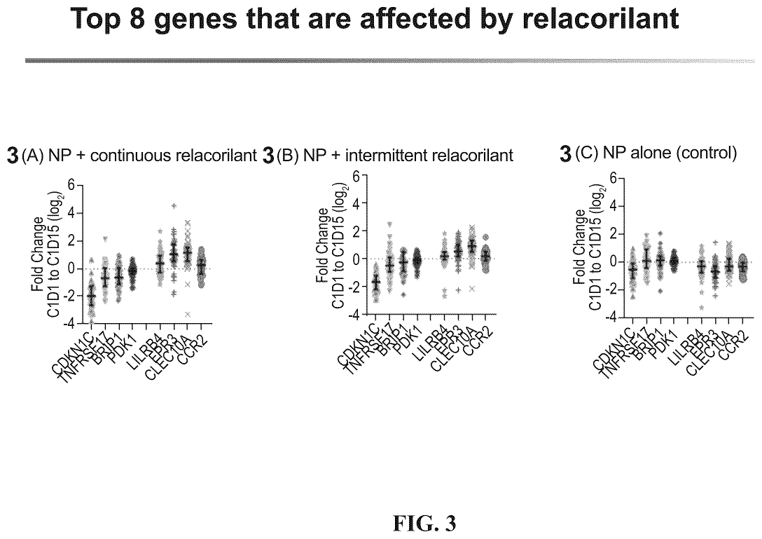

To determine if changes in a set of genes were associated with relacorilant activity, the fold change in each gene from cycle 1 day 1 to cycle 1 day 15 was first calculated and log 2 transformed. The resulting data were analyzed by machine learning to determine if there were differences in gene changes in the two relacorilant+NP arms combined vs the NP alone arm. Cross-validated random forest methods confirmed that gene changes, but not baseline gene values alone, could distinguish the groups with an ROC AUC of 0.945+/−0.038 (FIG. 2). The genes were then ranked based on significance in a t-test comparing fold change in relacorilant+nab-paclitaxel versus nab-paclitaxel alone. If the median fold change in a gene was greater in the nab-paclitaxel alone arm than in the relacorilant+nab-paclitaxel arms, this gene was considered a nab-paclitaxel-responsive gene and not analyzed further. From the resulting list, the top 4 induced genes and the top 4 suppressed genes were identified in the continuous relacorilant+NP arm (FIG. 3A). The intermittent relacorilant+nab-paclitaxel arm of the randomized ovarian phase 2 study showed a similar pattern with a smaller effect size (FIG. 3B), likely due to the SGRM washout during cycle 1 days 10-13 followed by just a single dose of relacorilant at cycle 1 day 14 prior to pharmacodynamic sampling. These genes are affected by relacorilant while the nab-paclitaxel effects are either opposite or minimal (ie, significantly less) (FIG. 3C and TABLE 2)

TABLE 2
Descriptive Statistics for 8 genes associated with relacorilant activity
Median log₂ fold change values for each gene in each arm of the ovarian phase 2 study

| | CDKN1C | TNFRSF17 | BRIP1 | PDK1 | LILRB4 | FPR3 | CLEC10A | CCR2 |
|---|---|---|---|---|---|---|---|---|
| Nab-paclitaxel | −0.5251 | 0.08885 | 0.1257 | 0.08098 | −0.3144 | −0.6865 | −0.3062 | −0.3171 |
| Relacorilant + | −1.980 | −0.7060 | −0.6290 | −0.1794 | 0.3807 | 1.038 | 1.153 | 0.2514 |
| nab-paclitaxel | | | | | | | | |
| P-value | 2.3e−11 | 5.3e−5 | 0.00013 | 0.00038 | 2.5e−5 | 4.7e−13 | 3.6e−9 | 1.2e−5 |

The median log 2 fold change values for each gene in each arm of the ovarian phase 2 study are shown. The p-values represent adjusted T-tests comparing the fold changes in the intermittent and continuous relacorilant+nab-paclitaxel arms to the nab-paclitaxel arm.

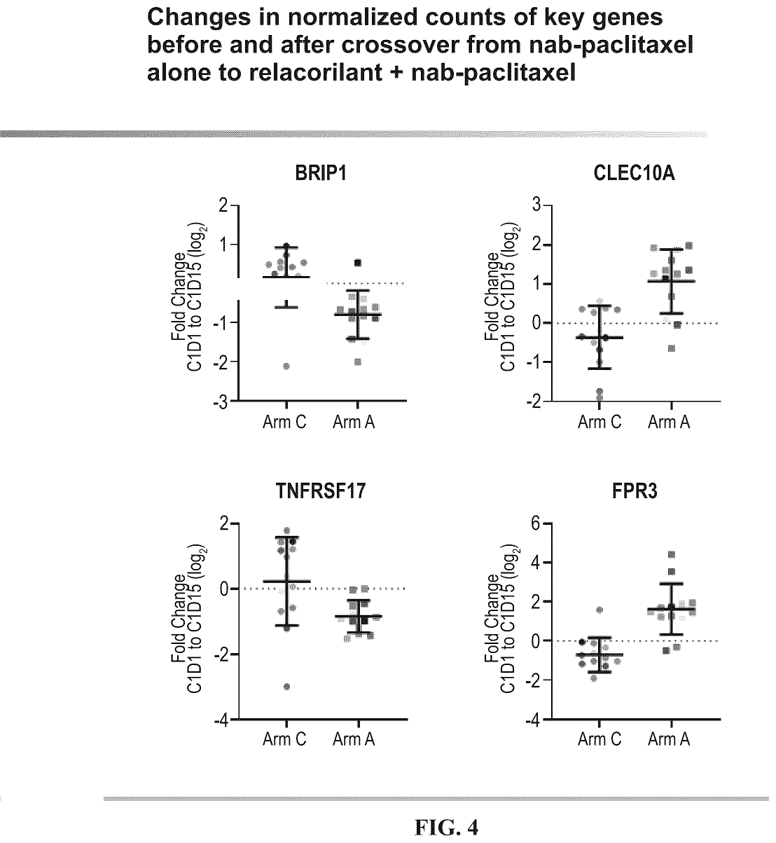

To validate this gene set, Applicant determined if these genes were altered after, but not before, crossover from nab-paclitaxel alone to nab-paclitaxel+continuous relacorilant. The 8 key genes were identified based on changes within each treatment arm in distinct subjects. In the crossover analysis, changes within each study subject before and after the crossover were assessed for each gene. Paired (by subject) T-tests were conducted to identify the genes that were most differentially modulated pre-crossover vs post crossover in 13 subjects. The top genes were FPR3 (adj P=0.00015), CLEC10A (adj P=0.00027), TNFRSF17 (adj P=0.00047), and BRIP1 (adj P=0.0029) (FIG. 4). (The term “adj” means that Benjamini-Yekutieli false discovery corrections were applied.) Further, the direction of the changes was consistent with the prior analysis. Specifically, BRIP1 and TNFRSF17 were suppressed after crossover while FPR3 and CLEC10A were induced after crossover.

### Gene Set is Consistently Modified by SGRMs Across Distinct Clinical Studies

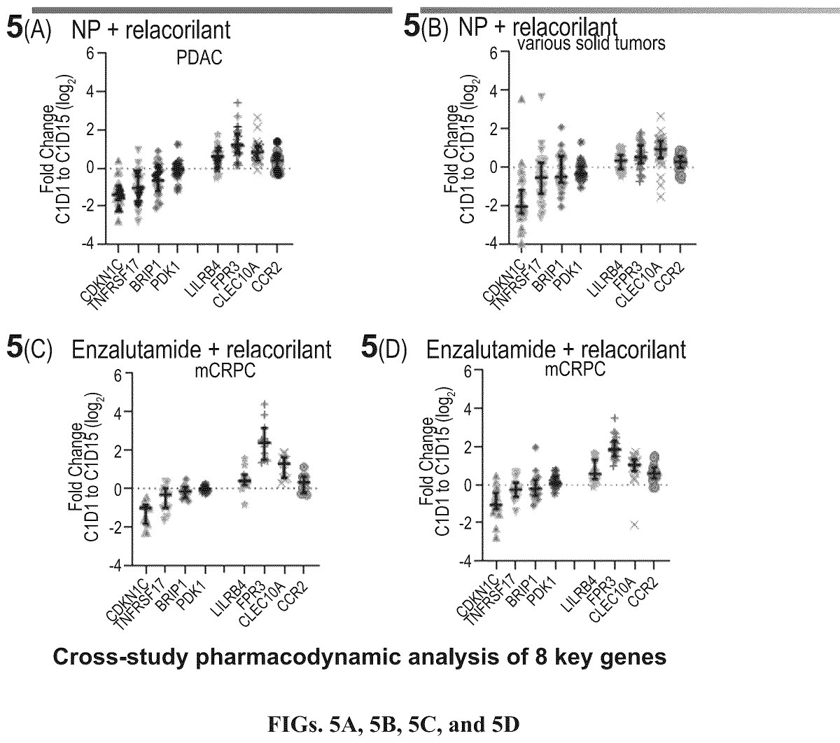

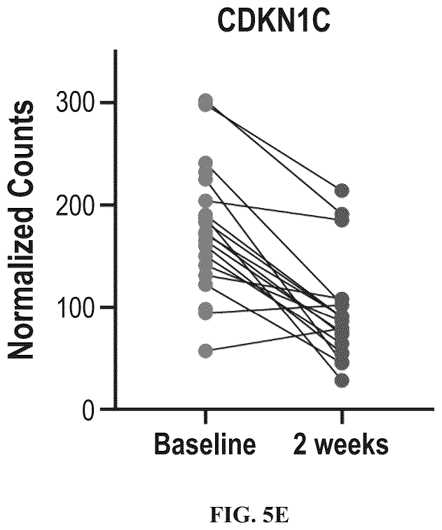

The 8 genes identified above were assessed in additional clinical trials of SGRMs to determine whether they are robust markers of SGRM activity (FIG. 5). Individual studies inevitably contain confounding variables, such as disease specific phenomena, concomitant medications, and unique toxicities (including hematological toxicities that can influence gene expression in whole blood, such as neutropenia). Identifying changes in these 8 genes across studies in distinct disease types, with distinct concomitant medications, and with different cytotoxic combination agents is thus a powerful confirmation that they are controlled by GR. First, a non-randomized phase 2 trial of continuous relacorilant+nab-paclitaxel in metastatic pancreatic ductal carcinoma (mPDAC) was assessed. Compared to the ovarian cancer phase 2 study in which this gene panel was identified, this PDAC study included both similar concomitant medications (i.e., G-CSF was mandated on days 2 and 9 of cycle 1 for patients receiving relacorilant in both studies) and combination agent (nab-paclitaxel). Across the 8-gene panel, a similar pattern of suppression/induction as in the ovarian cancer study was observed, confirming that these gene changes are not specific to patients with ovarian cancer (FIG. 5A). Similarly, a phase 1 dose-escalation study of relacorilant+nab-paclitaxel confirmed similar gene changes (FIG. 5B). Two metastatic castration resistant prostate cancer studies assessing the combination of continuous exicorilant+enzalutamide or continuous relacorilant+enzalutamide also revealed the same changes in the 8 key genes (FIG. 5C, D). This provides additional confirmation that changes in these genes are not driven by nab-paclitaxel and shows they are useful in assessing the activity of related SGRM drug candidates. FIG. 5E illustrates CDKN1C expression levels at baseline and after two weeks of exicorilant administration in prostate cancer patients receiving enzalutamide and exicorilant. CDKN1C is an established glucocorticoid-inducible gene with important roles in regulating cell growth. Expression levels of CDKN1C were suppressed after 2 weeks of dosing with exicorilant 240 mg+enzalutamide 160 mg (paired T-test P<0.0001).

### CLEC10a is a Marker of Glucocorticoid Receptor Activity

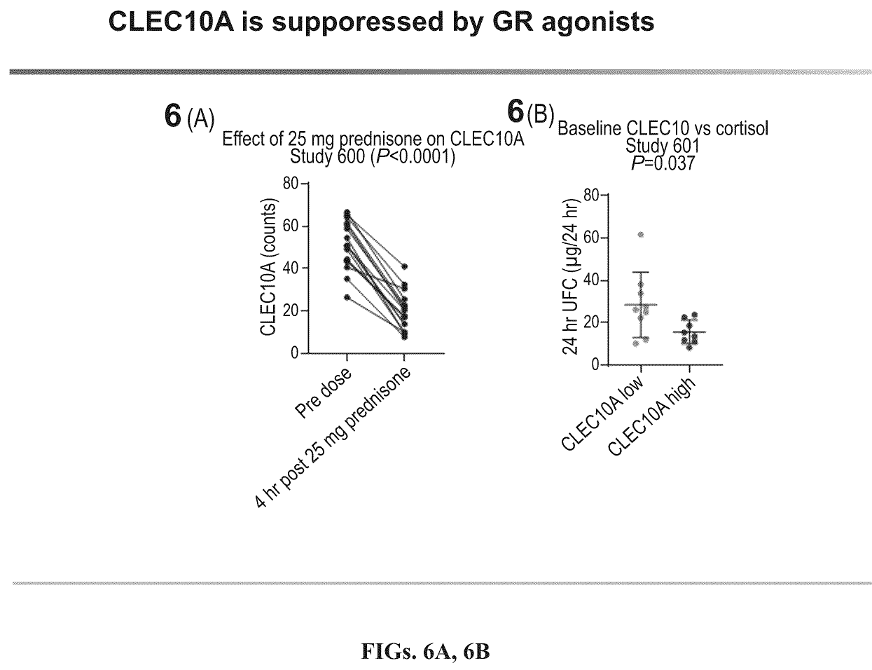

To determine if the 8 identified genes are more broadly associated with GR activity, the effects of a GR agonist (prednisone) on these genes were assessed. This is important in ruling out off-target effects associated with the chemical series to which relacorilant and exicorilant belong (fused azadecalins). CLEC10A was selected for further analysis because the prednisone effect (fold change of 0.37, adj P=6.4×10⁻⁸) was larger than the other 7 genes identified. (FIG. 6C). CLEC10A suppression by agonist (prednisone) is reciprocal to its induction by antagonist (SGRM).

As the primary endogenous GR agonist in humans is cortisol, the relationship between cortisol and CLEC10A expression was assessed. While serum cortisol is difficult to interpret due to its diurnal and ultradian variation, cortisol in urine pooled from a 24-hour period (24-hr urinary free cortisol [UFC]) is a more reliable assessment of systemic cortisol levels. Baseline urine was collected for 24 hr prior to cycle 1 day 1 in the exicorilant+enzalutamide mCRPC study and 24-hr UFC was quantified. In the subjects for which both baseline CLEC10A and 24-hr UFC were available, high and low CLEC10A groups were defined by baseline counts above or below the median CLEC10A counts. The subjects with low baseline CLEC10A had significantly higher 24-hr UFC (P=0.037) (FIG. 6D). Conversely, subjects with high baseline CLEC10A had lower 24-hr UFC. These observations suggest that cortisol suppresses CLEC10A. Thus, CLEC10A expression in whole blood was induced by relacorilant, unaffected by nab-paclitaxel, and suppressed by the GR agonists prednisone and cortisol.

CLEC10A Induction by Relacorilant is Associated with Improved Overall Survival in Patients with Ovarian Cancer

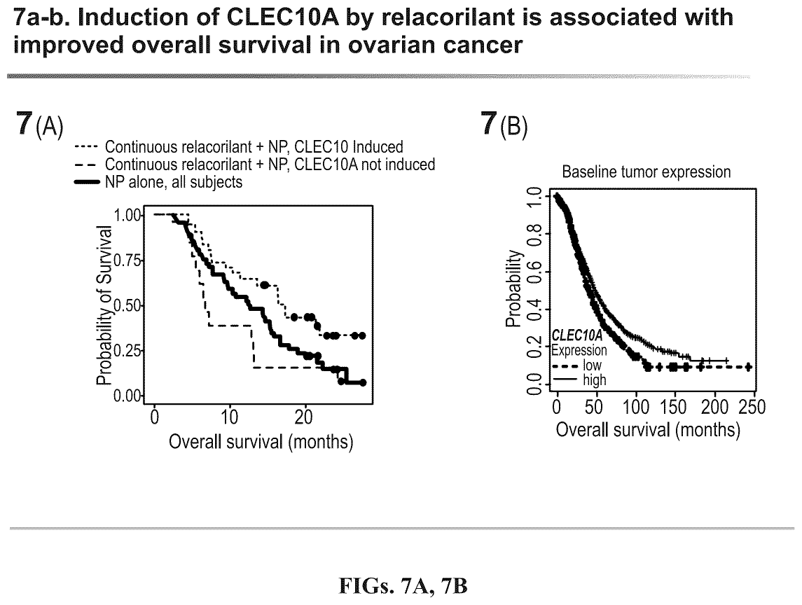

CLEC10A induction by relacorilant was pronounced in the ovarian phase 2 study, so Applicant next investigated whether this induction was associated with overall survival. The continuous relacorilant+NP arm was selected for this analysis because the pharmacodynamic sampling time was optimal for assessment of gene changes due to relacorilant. The fold change in CLEC10A was split into two groups representing the lower tertile vs upper two tertiles (based on a log 2 fold change cutoff of 0.63) in this treatment arm. Subjects with CLEC10A induction after relacorilant+NP experience significantly longer overall survival (median 17.2 vs 6.6 mo, HR=0.39, cox PH P=0.0135) (“PH” means “proportional hazard; see Cox, (1972). “Regression Models and Life-Tables”. Journal of the Royal Statistical Society, Series B. 34 (2): 187-220) than those without (FIG. 7A). Overall survival of all subjects (regardless of CLEC10A induction) treated with NP alone is shown as a comparator (FIG. 7A). Subjects with CLEC10A induction after relacorilant+NP experience significantly longer overall survival as compared to all subjects receiving NP alone (median 17.2 vs 12.2 mo, HR=0.55, cox PH P=0.028).

To better understand the functional role of CLEC10A and its relevance to ovarian cancer, publicly available databases were analyzed for association of tumor CLEC10A expression with prognosis [Nagy et al., Scientific Reports, 11, 6047 (2021)]. Across a large set of ovarian tumors (N=1656), the range of CLEC10A counts was 1-1173; and the lower tertile (<44 counts) was again defined as ‘low” CLEC10A in this analysis. Consistent with previous reports, high baseline tumor expression of CLEC10A was favorable and associated with longer OS in ovarian cancer (HR=0.8, log rank P=0.00084) (FIG. 7B).

### Expression of CLEC10A in Dendritic Cells

To understand the source of CLEC10A in our whole blood RNA profiling and the ovarian tumor database, the cell types associated with CLEC10A expression were investigated next. For each of the 8 genes in our GR-controlled gene set, the relative expression in common cell types was compared (Table 3) [Papatheodorou et al., Nucleic Acids Research, 46, D246-D251 (2018)]. CLEC10A expression was most pronounced in dendritic cells. In our whole blood analysis, the four genes with which log 2 transformed CLEC10A expression was most positively correlated are FPR3 (spearman R=0.72), CCR2 (spearman R=0.57), LILRB4 (spearman R=0.53), and CD86 (spearman R=0.47). FPR3, CCR2, and LILRB4 also had high expression in dendritic cells (Table 3). CD86, which was correlated with CLEC10A but not consistently GR modulated in our data sets (not shown), is a well-described marker of dendritic cells [Collin et al., Immunology, 140, 22-30 (2013)].

TABLE 3
Expression of CLEC10A and other GR target genes in common blood cell types

| | CDKN1C | TNFRSF17 | BRIP1 | PDK1 | LILRB4 | FPR3 | CLEC10A | CCR2 |
|---|---|---|---|---|---|---|---|---|
| CD14-positive, CD16- | 0.162602 | 0 | 0.008251 | 0.033133 | 0.04698 | 0.002671 | 0.172414 | 0.044843 |
| negative classical | | | | | | | | |
| monocyte | | | | | | | | |
| macrophage | 0.01626 | 0 | 0.024752 | 0.018072 | 0.302013 | 0.142476 | 0.015674 | 0 |
| inflammatory macrophage | 0 | 0 | 0.024752 | 0.012048 | 0.501119 | 0.356189 | 0 | 0 |
| conventional dendritic cell | 0 | 0 | 0.024752 | 0.009036 | 0.073826 | 0.436331 | 0.446708 | 0.006406 |
| CD4-positive, alpha-beta | 0.04065 | 0 | 0.066007 | 0.111446 | 0 | 0 | 0 | 0.003844 |
| thymocyte | | | | | | | | |
| CD8-positive, alpha-beta | 0.020325 | 0 | 0.041254 | 0.069277 | 0 | 0 | 0 | 0 |
| thymocyte | | | | | | | | |
| CD3-positive, CD4- | 0.04065 | 0.034483 | 0.09901 | 0.039157 | 0 | 0 | 0 | 0.003203 |
| positive, CD8-positive, | | | | | | | | |
| double positive thymocyte | | | | | | | | |
| CD38-negative naive B | 0 | 0.275862 | 0.007426 | 0.03012 | 0 | 0 | 0 | 0.003844 |
| cell | | | | | | | | |
| Memory B cell | 0 | 0.344828 | 0 | 0.009036 | 0 | 0 | 0 | 0.012812 |
| Class switched memory B | 0 | 0.344828 | 0.024752 | 0.009036 | 0 | 0 | 0 | 0.005766 |
| cell | | | | | | | | |
| CD4-positive, alpha-beta T | 0 | 0 | 0.016502 | 0.150602 | 0 | 0 | 0 | 0.003203 |
| cell | | | | | | | | |
| CD8-positive, alpha-beta T | 0.010163 | 0 | 0.016502 | 0.063253 | 0 | 0 | 0 | 0 |
| cell | | | | | | | | |
| Alternatively activated | 0.014228 | 0 | 0.00495 | 0.012048 | 0.073826 | 0.035619 | 0 | 0 |
| macrophage | | | | | | | | |
| Central memory CD4- | 0.010163 | 0 | 0.008251 | 0.021084 | 0 | 0 | 0 | 0.134529 |
| positive, alpha-beta T cell | | | | | | | | |
| Effector memory CD4- | 0 | 0 | 0.005776 | 0.006024 | 0 | 0 | 0 | 0.172966 |
| positive, alpha-beta T cell | | | | | | | | |
| Central memory CD8- | 0.012195 | 0 | 0.008251 | 0.006024 | 0 | 0 | 0 | 0.076874 |
| positive, alpha-beta T cell | | | | | | | | |
| Effector memory CD8- | 0.012195 | 0 | 0.016502 | 0.012048 | 0 | 0 | 0 | 0.140935 |
| positve, alpha-beta T cell | | | | | | | | |
| Regulatory T cell | 0.010163 | 0 | 0.016502 | 0.027108 | 0 | 0 | 0 | 0.16656 |
| Cytotoxic CD56-dim | 0 | 0 | 0.008251 | 0.012048 | 0 | 0 | 0 | 0 |
| natural killer cell | | | | | | | | |
| erythroblast | 0.060976 | 0 | 0.107261 | 0.033133 | 0 | 0.026714 | 0.344828 | 0.115311 |
| CD34-negative, CD41- | 0.060976 | 0 | 0.107261 | 0.087349 | 0 | 0 | 0 | 0 |
| positive, CD42-positive | | | | | | | | |
| megakaryocyte cell | | | | | | | | |
| Mature eosinophil | 0.020325 | 0 | 0.024752 | 0.063253 | 0 | 0 | 0.004702 | 0.057655 |
| Mature neutrophil | 0.162602 | 0 | 0.008251 | 0.054217 | 0 | 0 | 0 | 0 |
| Segmented neutrophil of | 0.020325 | 0 | 0.016502 | 0.033133 | 0.002237 | 0 | 0.007837 | 0.044843 |
| bone marrow | | | | | | | | |
| Neutrophilic | 0.101626 | 0 | 0.090759 | 0.036145 | 0 | 0 | 0 | 0.006406 |
| metamyelocyte | | | | | | | | |
| Endothelial cell of | 0.121951 | 0 | 0.107261 | 0.021084 | 0 | 0 | 0 | 0 |
| umbilical vein | | | | | | | | |
| (proliferating) | | | | | | | | |
| Endothelial Cell of | 0.101626 | 0 | 0.115512 | 0.021084 | 0 | 0 | 0.007837 | 0 |
| umbilical vein (resting) | | | | | | | | |

For each of the 8 GR target genes identified in the whole blood analysis, the normalized expression in each blood cell type is listed (bolded is high relative expression (≥0.02), unbolded is low relative expression (<0.02)).

### DISCUSSION

A set of 8 genes was identified that provide reliable markers of SGRM activity, and provide improved reliability as markers of SGRM activity when levels of two or more of these genes are considered together. Further, the CLEC10A gene is inversely regulated by GR agonists and antagonists (i.e., prednisone decreases and SGRMs increase CLEC10A levels). Baseline CLEC10A is associated with endogenous cortisol levels as well. Induction of CLEC10A in whole blood is associated with longer OS (overall survival) in patients receiving the combination of relacorilant+NP. Thus, change from baseline in whole blood CLEC10A RNA is both a pharmacodynamic biomarker, useful in determining an active dose of SGRM, and a predictive biomarker, useful in identifying patients likely to have longer survival after SGRM+NP therapy. Baseline whole blood CLEC10A RNA may be useful in identifying patients with elevated cortisol activity, and baseline tumor CLEC10A appears to be prognostic in multiple solid tumors.

The effects reported here on CLEC10A and its correlates (FPR3, CCR2, LILRB4, and CD86) likely represent a change in abundance of a set of dendritic cells. An alternative interpretation of such RNA changes would be that the cellular composition of a biospecimen remains consistent after treatment and genes are uniformly induced or suppressed across most or all of those cells. CLEC10A (called the type II GalNAc-specific, C-type lectin domain family 10 member A, CD301, macrophage galectin-type lectin, or MGL) is a marker of a subset of DC2A and DC2B dendritic cells (DCs) [Heger et al., Frontiers in Immunology, 9, 1-16 (2018); Hoober et al., Frontiers in Immunology, 10, 1-8 (2019)]. It is not expressed in CD16+DCs, CD141+DCs, and pDCs [Heger et al., Frontiers in Immunology, 9, 1-16 (2018)]. These findings provide additional detail to previous reports that demonstrate a clear role for GR agonists suppressing dendritic cells systemically [Olnes et al., Scientific Reports, 6, 23002 (2016); Shodell et al., Lupus, 12, 222-230 (2003)].

The association of CLEC10A induction with longer OS after relacorilant+NP confirms an important link between systemic SGRM activity and tumor response. CLEC10A binds glycosylated antigens such as the Tn-antigen [Zizzari et al., Journal of Immunology Research, 1-8 (2015)], so its increase may either reflect elevated antigen release by apoptotic tumor cells or an enhanced immune response to that antigen. Previous reports had suggested a prognostic benefit of high CLEC10A tumor expression [He et al., Journal of Cellular and Molecular Medicine, 25, 3391-3399 (2021); Zhou et al., Cellular Immunology, 372, 104472 (2022); Tang et al., PREPRINT (Version 1) available at Research Square. Downloaded from https://doi.org/10.21203/rs.3.rs-895659/v1 (2021)]. This suggests the CLEC10A expressing cells are beneficial in limiting tumor growth under various circumstances and treatments. The present new findings with SGRM demonstrate that increasing systemic CLEC10A is also associated with a survival benefit for patients with ovarian cancer receiving relacorilant+NP.

Systemic biomarkers of SGRMs have multiple applications. First, a pharmacodynamic assay could guide selection of an active dose of SGRMs. This is particularly useful for a competitive antagonist whose endogenous competitor (cortisol) is dynamic (e.g., whose levels may vary over time). While pharmacodynamics of cortisol synthesis inhibitors (i.e., metyrapone, mitotane, or ODM-208) can be assessed by measuring cortisol, SGRMs do not directly alter cortisol levels and thus require a biomarker downstream of GR. Second, such biomarkers may help identify patients with elevated cortisol activity, whether subclinical or as part of the difficult-to-diagnose Cushing's syndrome. More broadly, it could elucidate the specific function of GR systemically and define a new subset of GR-targeted dendritic cells. While the effects of glucocorticoids were initially described in 1924, our findings provide new clinically-relevant insights into the impact of GR on the human body

All patents, patent publications, publications, and patent applications cited in this specification are hereby incorporated by reference herein in their entireties as if each individual publication or patent application were specifically and individually indicated to be incorporated by reference. In addition, although the foregoing has been described in some detail by way of illustration and example for purposes of clarity of understanding, it will be readily apparent to those of ordinary skill in the art in light of the teachings herein that certain changes and modifications may be made thereto without departing from the spirit or scope of the appended claims.

1. A method of identifying a patient with cancer likely to benefit from administration of a selective glucocorticoid receptor modulator (SGRM) and a cancer therapeutic, and treating said identified cancer patient, the method comprising:
measuring a baseline level in a sample obtained from the cancer patient, of an RNA encoding a gene selected from CDKN1C, TNFRSF17, BRIP1, PDK1, CLEC10A, FPR3, CCR2, LILRB4, and CD86;
administering a SGRM and a cancer therapeutic to said cancer patient;
measuring a change in the level, as compared to said baseline level, in a sample obtained from the cancer patient following said administration of a SGRM and a cancer therapeutic, of said RNA encoding a gene selected from CDKN1C, TNFRSF17, BRIP1, PDK1, CLEC10A, FPR3, CCR2, LTLRB4, and CD86;
Wherein a change of said RNA level that is at least 40% as compared to the baseline level identifies the cancer patient as likely to benefit from further administration of said SGRM and said cancer therapeutic,
And continuing said administration of the SGRM and the cancer therapeutic to said patient identified as likely to benefit from said treatment,
Thereby treating said identified cancer patient.

2. The method of claim 1, wherein the benefit to the identified cancer patient comprises increased survival.

3. The method of claim 1, wherein the identified cancer patient suffers from a cancer selected from ovarian cancer, pancreatic cancer, and prostate cancer.

4. The method of claim 1, wherein the SGRM is
a) relacorilant, wherein relacorilant has the chemical name (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, and has the following structure: 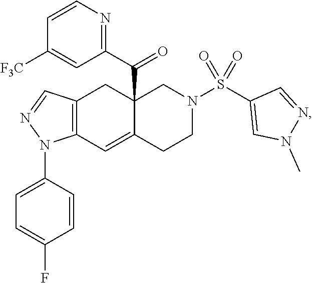 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]
Or
b) exicorilant, wherein exicorilant has the chemical name ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, and has the following structure:

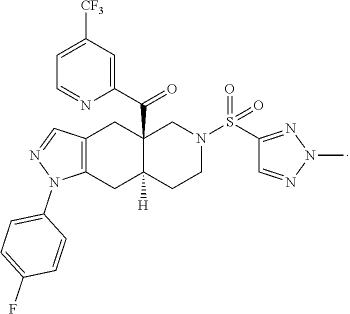 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

5. The method of claim 1, wherein the cancer therapeutic is a taxane selected from paclitaxel and nab-paclitaxel.

6. The method of claim 5, wherein the SGRM is relacorilant and the taxane is nab-paclitaxel.

7. The method of claim 5, wherein the SGRM is exicorilant and the taxane is nab-paclitaxel.

8. The method of claim 1, wherein the cancer therapeutic is an antiandrogen.

9. The method of claim 8, wherein the antiandrogen is enzalutamide.

10. The method of claim 9, wherein the SGRM is relacorilant.

11. The method of claim 9, wherein the SGRM is exicorilant.

12. The method of claim 1, wherein said sample obtained from the cancer patient is a whole blood sample.

13. The method of claim 1, wherein said RNA encodes CLEC10A.

14. A method of identifying an active dose of a selective glucocorticoid receptor modulator (SGRM) in a patient suffering from cancer, the method comprising:
measuring a baseline level, in a sample obtained from the patient, of an RNA encoding a gene selected from CDKN1C, TNFRSF17, BRIP1, PDK1, CLEC10A, FPR3, CCR2, LILRB4, and CD86;
administering a dose of a SGRM and of a cancer therapeutic to said cancer patient;
then
measuring, in a sample obtained from the patient, a change in the level, as compared to the baseline level, of an RNA encoding a gene selected from CDKN1C, TNFRSF17, BRIP1, PDK1, CLEC10A, FPR3, CCR2, LILRB4, and CD86;
Wherein a dose of said SGRM that results in a) a decrease in the level of an RNA encoding CDKN1C, TNFRSF17, BRIP1, or PDK1 or b) an increase in the level of an RNA encoding CLEC10A, FPR3, CCR2, LILRB4, and CD86 that is at least 40% as compared to the corresponding baseline level identifies the SGRM dose as an active dose.

15. The method of claim 14, comprising administering a plurality of doses of said SGRM and of said cancer therapeutic to said cancer patient, said plurality of SGRM doses each having a different amount of SGRM than the other SGRM doses.

16. The method of claim 14, wherein the SGRM dose of the plurality of SGRM doses which comprises the least amount of said SGRM that results in a) a decrease in the level of an RNA encoding CDKN1C, TNFRSF17, BRIP1, or PDK1 or b) an increase in the level of an RNA encoding CLEC10A, FPR3, CCR2, LILRB4, and CD86 that is at least 40% as compared to the corresponding baseline level is identified as the active SGRM dose.

17. The method of claim 14, wherein the cancer therapeutic is a taxane.

18. The method of claim 14, wherein the cancer therapeutic is an antiandrogen.

19. The method of claim 14, wherein the cancer patient suffers from a cancer selected from ovarian cancer, pancreatic cancer, and prostate cancer.

20. The method of claim 14, wherein the SGRM is
a) relacorilant, wherein relacorilant has the chemical name (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridin-2-yl)methanone, and has the following structure: 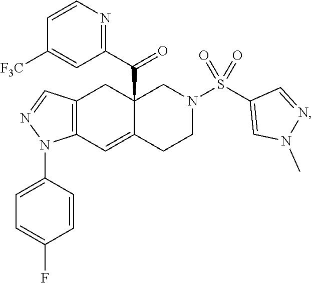 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]
Or
b) exicorilant, wherein exicorilant has the chemical name ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, and has the following structure:

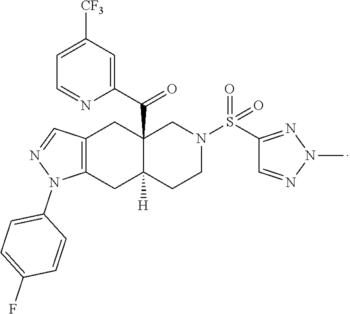 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

21. The method of claim 20, wherein the taxane is selected from paclitaxel or nab-paclitaxel.

22. The method of claim 14, wherein the SGRM is relacorilant and the cancer therapeutic is nab-paclitaxel.

23. The method of claim 14, wherein the SGRM is exicorilant and the cancer therapeutic is nab-paclitaxel.

24. The method of claim 14, wherein the RNA levels are measured in whole blood samples obtained from the patient.

25. The method of claim 14, wherein the RNA encodes CLEC10A.

26. The method of claim 14, further comprising treating said cancer patient, comprising: administering to the cancer patient a further dose of said cancer therapeutic and a further active dose of the SGRM, said further active dose consisting of an SGRM dose having an SGRM amount equal to, or greater than, said SGRM dose identified by the method, effective to treat the cancer patient.

27. A method of identifying an active dose of a selective glucocorticoid receptor modulator (SGRM) in a patient with Cushing's syndrome, the method comprising:
measuring a baseline level, in a sample obtained from said patient with Cushing's syndrome, of an RNA encoding a gene selected from CDKN1C, TNFRSF17, BRIP1, PDK1, CLEC10A, FPR3, CCR2, LILRB4, and CD86;
administering a dose of a SGRM to the patient with Cushing's syndrome; then
measuring, in a sample obtained from the patient with Cushing's syndrome, a change in the level, as compared to said baseline level, of an RNA encoding a gene selected from CDKN1C, TNFRSF17, BRIP1, PDK1, CLEC10A, FPR3, CCR2, LTLRB4, and CD86,
Wherein a dose of said SGRM that results in a) a decrease in the level of RNA encoding CDKN1C, TNFRSF17, BRIP1, PDK1, or b) an increase in the level of RNA encoding CLEC10A, FPR3, CCR2, LTLRB4, and CD86 that is at least 40% as compared to the baseline level is identified as an active dose of the SGRM.

28. The method of claim 27, comprising administering a plurality of doses of said SGRM to said patient with Cushing's syndrome, said plurality of SGRM doses each having a different amount of SGRM than the other SGRM doses.

29. The method of claim 27, wherein the SGRM dose of the plurality of SGRM doses which comprises the least amount of said SGRM that results in a) a decrease in the level of an RNA encoding CDKN1C, TNFRSF17, BRIP1, or PDK1 or b) an increase in the level of an RNA encoding CLEC10A, FPR3, CCR2, LILRB4, and CD86 that is at least 40% as compared to the corresponding baseline level is identified as the active SGRM dose.

30. The method of claim 27, further comprising treating said patient with Cushing's, comprising: administering to the patient with Cushing's syndrome a further active dose of the SGRM, said further active dose consisting of an SGRM dose having an SGRM amount equal to, or greater than, said SGRM dose identified by the method, effective to treat the patient with Cushing's syndrome.
